(19) United States
(12) Patent Application Publication
(10) Pub. No.: US 2023/0201318 A1
(43) Pub. Date: Jun. 29, 2023

(54) METHODS FOR TREATING HYPOPHOSPHATASIA IN CHILDREN AND ADOLESCENTS

(71) Applicant: Alexion Pharmaceuticals, Inc., Boston, MA (US)

(72) Inventors: Kenji FUJITA, Millburn, NJ (US); Marc VALLEE, Concord, MA (US); Dawn PHILLIPS, Chapel Hill, NC (US)

(21) Appl. No.: 18/057,483

(22) Filed: Nov. 21, 2022

Related U.S. Application Data
(63) Continuation of application No. 16/309,619, filed on Dec. 13, 2018, now abandoned.

(51) Int. Cl. A61K 38/46; A61P 19/00; A61B 5/11; A61K 9/00

(52) U.S. Cl. CPC A61K 38/465; A61P 19/00; A61B 5/112; A61K 9/0019; C12Y 301/03001

(57) ABSTRACT

The disclosure features methods for treating hypophosphatasia (HPP) in a patient (e.g., a child or an adolescent having HPP) exhibiting gait impairments or decreased walking ability by administering a soluble alkaline phosphatase (sALP) to the patient and assessing improvement in the gait impairment using a modified Performance-Oriented Mobility Assessment—Gait (mPOMA-G) analysis and score.

### INCORPORATION OF SEQUENCE LISTING

The instant application contains a Sequence Listing, which has been submitted electronically in XML format and is hereby incorporated by reference in its entirety. The XML copy, created on Nov. 17, 2022, is named 50694-061002_Sequence_Listing_11_17_22_ST26.XML and is 27,810 bytes in size.

### FIELD

The disclosure relates to methods for treating hypophosphatasia (HPP).

### BACKGROUND

Hypophosphatasia (HPP) is a rare, heritable skeletal disease with an incidence of 1 per 100,000 births for the most severe forms of the disease. The disorder results from loss-of-function mutations in the gene coding for tissue-nonspecific alkaline phosphatase (TNSALP). HPP exhibits a remarkable range of symptoms and severity in children adolescents, which may include rickets and proximal muscle weakness. Due to physical impairments associated with HPP, children and adolescent afflicted with HPP often exhibit decreased mobility relative to healthy peers. Mobility is a complex function requiring integration of strength, balance, and coordination, all of which are affected by HPP. Children and adolescents with HPP have functional deficits in mobility including ambulation difficulties, weakness (e.g., proximal muscle weakness), shortened stature, and an inability to perform activities of daily living.

There exists a need for methods that can be used to monitor and guide the treatment of HPP in patients with mobility impairments, particularly gait defects, such as children and adolescents.

### SUMMARY

Disclosed are (1) methods to identify (a) children having hypophosphatasia (HPP; e.g., children having HPP of about 5 to about 12 years of age) and (b) adolescents having hypophosphatasia (e.g., adolescents having HPP of about 12 to about 15 years of age) for treatment with a soluble alkaline phosphatase (sALP; e.g., SEQ ID NO: 1), and (2) treatment of such patients identified in (1) and (2) with an sALP. The modified Performance-Oriented Mobility Assessment Gait (mPOMA-G) is a useful metric to evaluate the need for or the efficacy of treatment using an sALP. The method includes the use of the mPOMA-G to assess treatment efficacy using an sALP for a patient having HPP, in which improvements relative to a certain score or value demonstrate that the sALP is effective for treating HPP.

The methods can further include the use of the mPOMA-G in combination with one or more other metrics (e.g., the Six Minute Walk Test (6MWT), Child Health Assessment Questionnaire (CHAO), and/or Pediatric Outcomes Data Collection Instrument (PODCI)) to assess treatment efficacy using an sALP for a patient having HPP, in which improvements relative to a certain score or value demonstrate that the sALP is effective for treating HPP. Additionally, the methods can further include changing the dosage of and/or the frequency of administration of the sALP (e.g., SEQ ID NO: 1) in order to determine the effective amount of the sALP to administer to a child or an adolescent having HPP.

A first aspect features a method of treating HPP in a patient of about 5 to about 15 years of age (e.g., a child or an adolescent having HPP, such as a patient having juvenile-onset HPP) having an average mPOMA-G score of about 8 or less (e.g., about 1, 2, 3, 4, 5, 6, 7, or 8), which includes administering a soluble alkaline phosphatase (sALP) to the patient at a dosage providing about 6 mg/kg/week of the sALP. In particular, the sALP includes an amino acid sequence having at least 95% sequence identity to the amino acid sequence of SEQ ID NO: 1 (e.g., asfotase alfa). Administration of the sALP (SEQ ID NO: 1) for a treatment period of at least one year results in an increase in the average mPOMA-G score of at least about 0.6 (e.g., about 0.7, about 0.8, or about 0.9), particularly at least about 1.0 or more (e.g., about 1, about 1.5, about 2.5, about 3, about 3.5, about 4, about 4.5, about 5, about 5.5, about 6, about 6.5, or about 7). For instance, administration of the sALP for a treatment period of at least one year results in an increase in the average mPOMA-G score of at least about 2.5 or more. The average mPOMA-G score of the patient can be determined relative to an average mPOMA-G score of an untreated subject (e.g., an HPP subject of about 5 to about 15 years of age) or a healthy subject (e.g., a healthy subject of about 5 to about 15 years of age).

The method can further include performing an mPOMA-G analysis, e.g., daily, one or more times per week (e.g., 2, 3, 4, 5, 6, or 7 times per week), weekly, one or more times per month (e.g., 2, 3, 4, 5, 6, 7, 8, 9, 10, 15, 20, 25, or more times per month), monthly, every other month, every three months, every six months, one or more times per year (e.g., 2, 3, 4, 5, 6, 7, 8, 9, 10, 11, 12, or more times per year), yearly, every two years, every three years, or a combination thereof (e.g., weekly combined with every three months, every six months, one or more times per year, or yearly).

For instance, the mPOMA-G analysis includes one or more gait assessments, such as trunk sway, walking stance, step length and height, step symmetry, and step continuity. In particular, trunk sway can include measuring at least one of marked sway, use of walking aids, arm abduction, trunk flexion, and excessive knee flexion; walking stance can include measuring distance of heels; step length and height can include measuring right swing foot, right foot clear, left swing foot, and/or left foot clear; step symmetry can include measuring right step length and left step length and comparing right step length to left step length; and step continuity can include measuring heel off in terminal stance on one foot at the same time as initial contact of heel strike on the opposite foot. For example, the mPOMA-G analysis is performed before or after administration of the sALP (e.g. SEQ ID NO: 1).

For instance, the sALP (e.g., SEQ ID NO: 1) is administered for a treatment period of at least two years, at least three years, at least four years, at least five years, at least six years, at least seven years, at least eight years, at least nine years, at least ten years, or longer (e.g., for the lifetime of the patient having HPP, such as a patient having juvenile-onset HPP). In particular, the average mPOMA-G score of the patient increases to about 7.5, about 8, about 8.5, about 9, about 9.5, about 10, about 10.5, about 11, about 11.5, or about 12 after administration of the sALP, such as to about 9 after administration of the sALP.

In the above aspects, the patient (e.g., a patient of about 5 to about 15 years of age having HPP, such as a patient having juvenile-onset HPP) prior to administration of the sALP can exhibit one or more gait impairments, including, but not limited to, reduced step length, reduced step continuity, reduced foot clearance, foot clearance that exceeds about 1 inch off of the surface, and widened stance. For example, the patient after administration of the sALP exhibits an improvement in one or more gait impairments, such as reduced step length, reduced foot clearance, and widened stance.

The patient having HPP can be a child or an adolescent. Additionally, the patient can be one that has not been previously administered the sALP (e.g., SEQ ID NO: 1).

As a result of the methods, the increase in the average mPOMA-G score is sustained throughout a period during which the patient (e.g., a patient of about 5 to about 15 years of age having HPP, such as a patient having juvenile-onset HPP) is treated with the sALP (e.g., at least one year, at least two years, at least three years, at least four years, at least five years, at least six years, at least seven years, at least eight years, at least nine years, at least ten years, or for the lifetime of the patient). For example, a rate of change per year of the average mPOMA-G score is about 1.0 or more (e.g., about 1, about 1.5, about 2.5, about 3, about 3.5, about 4, about 4.5, about 5, about 5.5, about 6, about 6.5, or about 7).

The method can further include performing at least one of a 6MWT, CHAQ, and/or a PODCI. For example, the patient (e.g., a patient of about 5 to about 15 years of age having HPP, such as a patient having juvenile-onset HPP) can exhibit an increase in activities of daily living (ADL) after administration of the sALP, e.g., in which the increase in ADL is determined from a CHAQ disability index score or PODCI transfer and mobility scale score of the patient. Additionally, the patient can exhibit an improvement in walking ability, e.g., in which the improvement in walking ability is determined from a 6MWT distance of the patient. For instance, the patient also exhibits decreased reliance on an assistive device (e.g., a wheelchair, braces, crutches, or an orthotic) for mobility after administration of the sALP.

Additionally, the method can further include determining sALP activity in at least one of serum and blood from the patient (e.g., a patient of about 5 to about 15 years of age having HPP, such as a patient having juvenile-onset HPP). For example, the sALP activity includes measuring at least one of phosphoethanolamine (PEA), inorganic pyrophosphate (PPi), and/or pyridoxal 5′-phosphate (PLP) in the serum and/or blood sample from the patient.

In the above aspects, the sALP (e.g., SEQ ID NO: 1) is administered to the patient (e.g., a patient of about 5 to about 15 years of age having HPP, such as a patient having juvenile-onset HPP) daily or weekly, such as twice a week, three times a week, four times a week, five times a week, six times a week, or seven times a week. For example, the sALP (e.g., SEQ ID NO: 1) is administered to the patient at a dosage of 2 mg/kg for administration three times a week, a dosage of 3 mg/kg for administration three times a week, or a dosage of 1 mg/kg for administration six times a week. Moreover, the sALP (e.g., SEQ ID NO: 1) is administered to the patient once daily and/or on consecutive or alternating days.

Additionally, the dose of the sALP is increased (e.g., from 6 mg/kg/day to 9 mg/kg/day) if the average mPOMA-G score does not increase by, e.g., at least about 1.0 or more (e.g., about 1, about 1.5, about 2.5, about 3, about 3.5, about 4, about 4.5, about 5, about 5.5, about 6, about 6.5, or about 7) after a treatment period of at least one year (e.g., at least two years, at least three years, at least four years, at least five years, at least six years, at least seven years, at least eight years, at least nine years, at least ten years, or for the lifetime of the patient). In particular, the dose of the sALP can be increased if the average mPOMA-G score does not increase by, e.g., at least about 2.5 or more (e.g., about 3, about 3.5, about 4, about 4.5, about 5, about 5.5, about 6, about 6.5, or about 7) after a treatment period of at least one year.

The patient (e.g., a patient of about 5 to about 15 years of age having HPP, such as a patient having juvenile-onset HPP) can exhibit one or more symptoms of HPP, which can include, but are not limited to, gait disturbance, bone deformity, joint pain, bone pain, bone fracture, muscle weakness, muscle pain, rickets, premature loss of deciduous teeth, incomplete bone mineralization, elevated blood and/or urine levels of phosphoethanolamine (PEA), elevated blood and/or urine levels of inorganic pyrophosphate (PPi), elevated blood and/or urine levels of pyridoxal 5′-phosphate (PLP), hypomineralization, rachitic ribs, hypercalciuria, short stature, HPP-related seizure, inadequate weight gain, craniosynostosis, and/or calcium pyrophosphate dihydrate crystal deposition. In particular, the patient exhibits an improvement in the one or more symptoms of HPP after administration of the sALP (e.g., SEQ ID NO: 1).

In the above aspects, the sALP (e.g., SEQ ID NO: 1) is formulated in a pharmaceutical composition, with at least one pharmaceutically acceptable carrier, such as saline (e.g., sodium chloride and sodium phosphate). For example, the at least one pharmaceutically acceptable carrier includes 150 mM sodium chloride and 25 mM sodium phosphate. Moreover, the pharmaceutical composition is formulated for subcutaneous, intramuscular, intravenous, oral, nasal, sublingual, intrathecal, or intradermal administration. In particular, the pharmaceutical composition is formulated for subcutaneous administration.

In the above aspects, the sALP (e.g., SEQ ID NO: 1) is physiologically active toward PEA, PPi, and PLP, catalytically competent to improve skeletal mineralization in bone, and/or is the soluble extracellular domain of an alkaline phosphatase. In particular, the sALP includes or consists of the amino acid sequence of SEQ ID NO: 1.

### Definitions

As used herein, “a” or “an” means “at least one” or “one or more” unless otherwise indicated. In addition, the singular forms “a,” “an,” and “the” include plural referents unless the context clearly dictates otherwise.

As used herein, “about” refers to an amount that is ±10% of the recited value and is preferably ±5% of the recited value, or more preferably ±2% of the recited value. For instance, the term “about” can be used to modify all dosages or ranges recited herein by ±10% of the recited values or range endpoints.

By “asfotase alfa” is meant a human TNSALP (hTNSALP) fusion protein formulated for the treatment of HPP. Asfotase alfa (STRENSIQ®, Alexion Pharmaceuticals, Inc.) is a fusion protein including a soluble glycoprotein of two identical polypeptide chains, in which each polypeptide chain includes amino acid residues 1-726 of SEQ ID NO: 1. The structure of each polypeptide chain includes the catalytic domain of hTNSALP, the human immunoglobulin Gi Fc domain, and a deca-aspartate peptide used as a bone targeting domain (the structure hTNSALP-Fc-D₁₀). The two polypeptide chains are covalently linked by two disulfide bonds. Asfotase alfa has been approved under the trade name STRENSIQ® in the United States, Europe, Japan, Canada, Israel, Australia, and Korea.

The terms “activities of daily living” or “ADL,” as used herein, refer to routine activities that healthy subjects perform on a daily basis without requiring assistance, such as functional mobility or transferring (e.g., walking), bathing and showering, dressing, self-feeding, and personal hygiene and grooming. As described herein, therapeutic compositions (e.g., compositions including a soluble alkaline phosphatase (sALP), such as asfotase alfa) can be administered to an HPP patient to improve the ability of the patient (e.g., an adolescent or a child) to perform ADL.

As used herein, “average” refers to a numerical value expressing the mean or median of a data set. The mean of a data set is calculated by dividing the sum of the values in the set by their number. The median of a data set is calculated by, e.g., arranging all values in the data set from the lowest value to the highest value and selecting the middle value. In a data set with an even number of values, the median may be calculated, e.g., as the mean of the two middle values.

The term “bone-targeting moiety,” as used herein, refers to an amino acid sequence of between 1 and 50 amino acid residues (such as 1, 2, 3, 4, 5, 6, 7, 8, 9, 10, 12, 14, 15, 16, 18, 20, 22, 24, 25, 26, 28, 30, 32, 34, 35, 36, 38, 40, 42, 44, 45, 46, 48, or 50 amino acid residues) in length having an affinity to bone matrix, such that the bone-targeting moiety, singularly, has an in vivo binding affinity to bone matrix that is about 10⁻⁶ M to about 10⁻¹⁵ M (e.g., 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, or 10⁻¹⁵ M). For example, the bone-targeting moiety can include a series of consecutive aspartate (D) and/or glutamate (E) residues of number “n,” in which n=1 to 50, e.g., n=3-30, e.g., 5-15, e.g., 1, 2, 3, 4, 5, 6, 7, 8, 9, 10, 11, 12, 13, 14, 15, 16, 17, 18, 19, 20, 21, 22 , 23, 24, 25, 26, 27, 28, 29, 30, 31, 32, 33, 34, 35, 36, 36, 38, 39, 40, 41, 42, 43, 44, 45, 46, 47, 48, 49, or 50.

The term “catalytically competent,” as used herein, refers to an sALP that hydrolyzes the bone mineralization inhibitor inorganic pyrophosphate (PPi) to provide inorganic phosphate (Pi), thereby decreasing the extracellular concentrations of PPi. A catalytically competent sALP improves skeletal mineralization in bone by regulating the concentration of PPi.

The term “dosage” refers to a determined quantity of an active agent (e.g., an sALP, such as asfotase alfa) calculated to produce a desired therapeutic effect (e.g., treatment of HPP, e.g., a reduction in one or many symptoms of HPP, such as a mobility impairment) that is administered to a patient at a particular frequency and/or in a defined amount. A dosage form can include an sALP, such as asfotase alfa, in association with any suitable pharmaceutical excipient, carrier, or diluent.

By “extracellular domain” is meant any functional extracellular portion of a native protein, e.g., alkaline phosphatase. In particular, an extracellular domain lacks a signal peptide.

By “Fc” is meant a fragment crystallizable region of an immunoglobulin, e.g., IgG-1, IgG-2, IgG-3, IgG-3 or IgG-4, including the CH2 and CH3 domains of the immunoglobulin heavy chain. Fc may also include any portion of the hinge region joining the Fab and Fc regions. The Fc can be of any mammal, including human, and may be post-translationally modified (e.g., by glycosylation). In a non-limiting example, Fc can be the fragment crystallizable region of human IgG-1 having the amino acid sequence of SEQ ID NO: 20.

By “fragment” is meant a portion of a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more, but less than the entire length of, a reference nucleic acid molecule or polypeptide. For example, a polypeptide fragment may contain 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 400, 500, 600, 700, or more amino acid residues of the reference polypeptide. Exemplary sALP fragments have amino acid residues 18-498, 18-499, 18-500, 18-501, 18-502, 18-503, 18-504, 18-505, 18-506, 18-507, 18-508, 18-509, 18-510, 18-511, or 18-512 of an ALP (e.g., SEQ ID NOs: 2-6), and may include additional C-terminal and/or N-terminal portions. Biological activity of such fragments can be tested in standard assays known in the art, e.g., by a non-compartmental analysis (NCA) to calculate pharmacokinetic parameters of the sALP fragment.

The term “gait,” as used herein, refers to the locomotion of a subject (e.g., a child or an adolescent having HPP) achieved through moving on foot, such as by walking, e.g., in which at least one foot is in contact with the ground at all times. In particular, human gait refers to bipedal and biphasic forward propulsion of the center of gravity of the human body over a surface, such as a floor. Normal gait for a human subject is when one limb typically provides support, while the other limb is advanced in preparation to support the other limb. The gait cycle (e.g., a stride) is composed of a swing phase and a stance phase, in which the stance phase includes initial double stance, single limb stance, and terminal double. The duration of a gait cycle is the interval between sequential initial surface contacts by the same limb.

The term “gait impairment,” as used herein, refers to abnormalities in normal gait of a subject (e.g., a human child or adolescent, such as a human child or adolescent with HPP) in which the subject exhibits deficiencies in the ability to stand and perform stepping movements for locomotion. Gait impairments can include, but are not limited to, reduced step length, reduced foot clearance, and a widened stance. For example, gait impairments can result in, e.g., waddling gait, in which the patient exhibits exaggerated alternation of lateral trunk movements with an exaggerated elevation of the hip, or steppage gait, in which the advancing leg is lifted high in order that the toes may clear the ground. Assessments of gait and gait impairments in a patient (e.g., a child or an adolescent having HPP) can include, e.g., determining the step length and height, step symmetry, step continuity, trunk sway, and walking stance of the patient.

The terms “hypophosphatasia” and “HPP,” as used herein, refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNSALP). HPP may be further characterized as infantile HPP, childhood HPP, perinatal HPP (e.g., benign perinatal HPP or lethal perinatal HPP), odonto-HPP, adolescent HPP, or adult HPP. For instance, “childhood HPP” describes a patient having HPP that is about 5 years of age to about 12 years of age, whereas “adolescent HPP” describes a patient having HPP that is about 12 years of age to about 18 years of age, such as a patient having HPP that is about 12 years of age to about 15 years of age. The age of onset of HPP, such as when the subject exhibits symptoms of HPP, can also be categorized as, e.g., perinatal-onset HPP, infantile-onset HPP, and juvenile-onset HPP (e.g., during childhood).

The term “HPP phenotype,” as used herein, refers to any one of gait disturbance, bone deformity, joint pain, bone pain, bone fracture, muscle weakness, muscle pain, rickets (e.g., defects in growth plate cartilage), premature loss of deciduous teeth, incomplete bone mineralization, elevated blood and/or urine levels of phosphoethanolamine (PEA), PPi, pyridoxal 5′-phosphate (PLP), hypomineralization, rachitic ribs, hypercalciuria, short stature, HPP-related seizure, inadequate weight gain, craniosynostosis, and/or calcium pyrophosphate dihydrate crystal deposition (CPPD) in joints leading to, e.g., chondrocalcinosis and premature death. Without being so limited, an HPP phenotype can be documented by one or more of gait deficits, as described herein; growth retardation with a decrease of long bone length (including but not limited to femur, tibia, humerus, radius, and/or ulna); a decrease of the mean density of total bone and a decrease of bone mineralization in bones such as femur, tibia, ribs and metatarsi, and phalange; a decrease in teeth mineralization; and a premature loss of deciduous teeth (e.g., aplasia, hypoplasia, or dysplasia of dental cementum). Without being so limited, correction or prevention of a bone mineralization defect may be observed by one or more of the following: a reduction in gait deficits, an increase of long bone length, an increase of mineralization in bone and/or teeth, a correction of bowing of the legs, a reduction of bone pain, and a reduction of CPPD crystal deposition in joints. The terms “modified Performance Oriented Mobility Assessment-Gait” and “mPOMA-G,” as used herein, refer to a modified version of the Performance Oriented Mobility Assessment—Gait (POMA-G), such as a POMA-G that is modified to provide improved sensitivity for gait impairments in POMA-G components that pertain to HPP patients (e.g., a child or an adolescent with HPP). The POMA is a validated tool that consists of 2 subtests: the POMA-G (Gait) and the POMA-B (Balance). See Tinetti et al. (Am J Med 80:429-434, 1986) for a description of the POMA-G; hereby incorporated by reference in its entirety. Gait performance of an HPP patient (e.g., a child or an adolescent with HPP) can be assessed using an mPOMA-G test, e.g., in which the POMA-G analysis has been modified to remove the less appropriate components, i.e., the initiation of gait and path components, of the POMA-G. For example, the mPOMA-G analysis can provide a total score of 12 points, similarly to the POMA-G components, of step length and height, step symmetry, step continuity, trunk sway, and walking stance, in which a score of 12 indicates no gait impairments, and lower scores indicate gait impairments.

By “pharmaceutically acceptable excipient, carrier, or diluent” is meant at least one excipient, carrier, or diluent, respectively, which is physiologically acceptable to the treated patient and which does not alter the therapeutic properties of an active agent (e.g., an sALP, such as asfotase alfa (SEQ ID NO: 1)) with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable excipients, carriers, and diluents, and their formulations, are known to those skilled in the art and described, e.g., in Remington's Pharmaceutical Sciences (20th edition), A. Gennaro, Ed., 2000, Lippincott, Williams & Wilkins, Philadelphia, PA. For instance, a pharmaceutically acceptable excipient, carrier, or diluent can include dibasic sodium phosphate, heptahydrate; monobasic sodium phosphate, monohydrate; and sodium chloride at a pH between 7.2 and 7.6.

By “pharmaceutical composition” is meant a composition containing an active agent, such as an sALP (e.g., an sALP comprising an amino acid sequence having at least 95% sequence identity to the amino acid sequence of SEQ ID NO: 1, such as asfotase alfa), as described herein, formulated with at least one pharmaceutically acceptable excipient, carrier, or diluent. The pharmaceutical composition may be manufactured or sold with the approval of a governmental regulatory agency as part of a therapeutic regimen for the treatment or prevention of a disease or event (e.g., HPP) in a patient (e.g., an HPP patient, such as a child or adolescent). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form. For example, an sALP (e.g., asfotase alfa; SEQ ID NO: 1) can be formulated as a pharmaceutical composition including dibasic sodium phosphate, heptahydrate; monobasic sodium phosphate, monohydrate; and sodium chloride at a pH between 7.2 and 7.6 for administration to a patient in, e.g., a weekly dosage ranging, e.g., from about 0.5 mg/kg/week to about 140 mg/kg/week, e.g., about 0.8 mg/kg/week to about 50 mg/kg/week, or about 1 mg/kg/week to about 10 mg/kg/week (e.g., about 6 or about 9 mg/kg/week).

The term “physiologically active,” as used herein, refers to an sALP that hydrolyzes phosphoethanolamine (PEA), inorganic pyrophosphate (PPi), and pyridoxal 5′-phosphate (PLP) to provide Pi, thereby decreasing extracellular concentrations of PEA, PPi, and PLP.

The terms “sALP,” “soluble alkaline phosphatase,” and “extracellular domain of an alkaline phosphatase” are used interchangeably and refer to a soluble, non-membrane bound ALP or a domain or a biologically active fragment of the soluble, non-membrane bound ALP. sALPs include, for example, an alkaline phosphatase lacking a C-terminal glycolipid anchor (GPI signal sequence, e.g., polypeptides including or consisting of the amino acid residues 18-502 of a human TNSALP (SEQ ID NOs: 2, 3, 4, 5, or 6)). In particular, a TNSALP may include, e.g., a polypeptide including or consisting of amino acid residues 1-485 of SEQ ID NO: 1, such as asfotase alfa, or a polypeptide variant having at least 95% sequence identity to the amino acid residues 1-485 of SEQ ID NO: 1. sALPs further include, for example, mammalian orthologs of human TNSALP, such as a rhesus TNSALP (SEQ ID NO: 7), a rat TNSALP (SEQ ID NO: 8), a canine TNSALP (SEQ ID NO: 9), a porcine TNSALP (SEQ ID NO: 10), a murine TNSALP (SEQ ID NO: 11), a bovine TNSALP (SEQ ID NOs: 12-14), or a feline TNSALP (SEQ ID NO: 15). sALPs also include soluble, non-membrane-bound forms of human PALP (e.g., polypeptides including or consisting of amino acid residues 18-502 of SEQ ID NOs: 16 or 17), GCALP (e.g., polypeptides including or consisting of amino acid residues 18-502 of SEQ ID NO: 18), and IALP (e.g., polypeptides including or consisting of amino acid residues 18-502 of SEQ ID NO: 19), and additional variants and analogs thereof that retain alkaline phosphatase activity, e.g., the ability to hydrolyze PR, such as variants having at least 90, 95, 97, or 99% sequence identity to any one of SEQ ID NOs: 7-19. An sALP, in particular, lacks the N-terminal signal peptide (e.g., aa 1-17 of SEQ ID NOs: 2-6, 8, 11-13, or 15 or aa 1-25 of SEQ ID NO: 7).

By “sALP fusion polypeptide” is meant a polypeptide having the structure Z-sALP-Y-spacer-X-Wₙ-V, Z-Wₙ-X-spacer-Y-sALP-V, Z-sALP-Y-Wₙ-X-spacer-V, and Z-Wₙ-X-sALP-Y-spacer-V. In particular, the sALP fusion polypeptide can be Z-sALP-Y-spacer-X-Wₙ-V or Z-Wₙ-X-spacer-Y-sALP-V, such as hTNSALP-Fc-D₁₀ (e.g., asfotase alfa; SEQ ID NO: 1). Any one of X, Y, Z, V, the spacer, and/or Wₙ can be absent or an amino acid sequence of at least one amino acid. For example, X, Y, Z, and V may be a dipeptide sequence (e.g., leucine-lysine or aspartic acid-isoleucine), such as a two residue linker at the Y position (e.g., leucine-lysine) and a two residue linker at the X position (e.g., aspartic acid-isoleucine). Spacers include, for example, a Fc region of an immunoglobulin, such as the amino acid sequence of SEQ ID NO: 20. Wₙ can be a bone-targeting moiety as defined herein, e.g., having a series of consecutive aspartate (D) or glutamate (E) residues, in which n=1 to 50, e.g., n=3-30, e.g., 5-15, e.g., 1, 2, 3, 4, 5, 6, 7, 8, 9, 10, 11, 12, 13, 14, 15, 16, 17, 18, 19, 20, 21, 22 , 23, 24, 25, 26, 27, 28, 29, 30, 31, 32, 33, 34, 35, 36, 36, 38, 39, 40, 41, 42, 43, 44, 45, 46, 47, 48, 49, or 50.

By “signal peptide” is meant a short peptide (5-30 amino acids long) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the Golgi apparatus. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal peptide can be one that is, for example, substantially identical to amino acid residues 1-17 of SEQ ID NOs: 2-6 or amino acid residues 1-25 of SEQ ID NO: 7.

As used herein, when a polypeptide or nucleic acid sequence is referred to as having “at least X% sequence identity” to a reference sequence, wherein “X” is a real number, it is meant that at least X percent of the amino acid residues or nucleotides in the polypeptide or nucleic acid, respectively, are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al., J. Mol. Biol. 215: 403-10, 1990). These and other alignment algorithms are accessible using publicly available computer software such as “Best Fit” (Smith and Waterman, Advances in Applied Mathematics, 482-489, 1981) as incorporated into GeneMatcher Plus (Schwarz and Dayhoff, Atlas of Protein Sequence and Structure, Dayhoff, M.O., Ed pp 353-358, 1979), BLAST, BLAST-2, BLAST-P, BLAST-N, BLAST-X, WU-BLAST-2, ALIGN, ALIGN-2, CLUSTAL, Megalign (DNASTAR), or other software/hardware for alignment. In addition, those skilled in the art can determine appropriate parameters for measuring alignment, including any algorithms needed to achieve optimal alignment over the length of the sequences being compared.

The terms “patient” or “subject” refer to a mammal, including, but not limited to, a human (e.g., a human having HPP, such as a child or adolescent) or a non-human mammal, such as a bovine, equine, canine, ovine, or feline.

“Parenteral administration,” “administered parenterally,” and other grammatically equivalent phrases, as used herein, refer to a mode of administration other than enteral and topical administration, usually by injection, and include, without limitation, subcutaneous, intradermal, intravenous, intranasal, intraocular, pulmonary, intramuscular, intra-arterial, intrathecal, intracapsular, intraorbital, intracardiac, intradermal, intrapulmonary, intraperitoneal, transtracheal, subcuticular, intraarticular, subcapsular, subarachnoid, intraspinal, epidural, intracerebral, intracranial, intracarotid, and intrasternal injection and infusion.

By “therapeutically effective amount” is meant an amount of an sALP (e.g., an sALP comprising an amino acid sequence having at least 95% sequence identity to the amino acid sequence of SEQ ID NO: 1, such as asfotase alfa) that is sufficient to substantially improve, treat, prevent, delay, suppress, or arrest at least one symptom of HPP (e.g., a mobility impairment, such as a gait impairment). A therapeutically effective amount of an sALP described herein may depend on the severity of HPP and the condition, weight, and general state of the patient and can be determined by an ordinarily-skilled artisan with consideration of such factors. A therapeutically effective amount of a composition described herein can be administered to a patient in a single dose or in multiple doses administered over a period of time.

By “treating,” “treat,” or “treatment” is meant the medical management of a patient (e.g., a child or an adolescent having HPP) with the intent to cure, ameliorate, stabilize, reduce the likelihood of, or prevent HPP and/or the management of a patient exhibiting or likely to have HPP, e.g., by administering a pharmaceutical composition, such as an sALP (e.g., an sALP comprising an amino acid sequence having at least 95% sequence identity to the amino acid sequence of SEQ ID NO: 1, such as asfotase alfa). Treatment can occur for a treatment period, in which an sALP is administered for a period of time (e.g., days, months, years, or longer) to treat a patient having HPP, such as a child or an adolescent with HPP that exhibits deficits in gait. This term includes active treatment directed toward the improvement of HPP; symptomatic treatment directed toward symptoms of HPP, such as deficits in gait; preventative treatment directed to minimizing the development of HPP, e.g., in a patient who does not yet have HPP, but who is susceptible to or at risk of developing HPP; and supportive treatment employed to supplement another specific therapy directed toward the improvement of HPP.

### BRIEF DESCRIPTION OF THE DRAWINGS

FIG. 1 is an image for gait assessment used in the key for the modified Performance-Oriented Mobility Assessment Gait (mPOMA-G) analysis of a child demonstrating step continuity.

FIG. 2 is an image used in the mPOMA-G analysis of a child demonstrating normal hip/knee flexion during swing for gait assessment.

FIGS. 3A-3C are graphs showing linear regression analysis of the mPOMA-G score relative to scores determined for the Childhood Health Assessment Questionnaire (CHAQ) Disability Index (FIG. 3A), Pediatric Outcomes Data Collection Instrument (PODCI) Transfer and Mobility Scale Normative Parent Score (FIG. 3B), and the Six Minute Walk Test (distance walked in meters) (6MWT; FIG. 3C) for children and adolescents with HPP treated with asfotase alfa.

FIG. 4 is a graph showing the rate of change per year in the mPOMA-G score from baseline to last assessment of asfotase alfa-treated HPP patients (n=8) compared with historical HPP controls (HCs; n=6). The P-value based on Wilcoxon rank sum test (exact method) comparing the median mPOMA-G for asfotase alfa-treated HPP patients and HCs is shown. The upper and lower bars represent the maximum and minimum values, respectively; the top and bottom of the box represent the 3rd and 1st quartiles, respectively; the line within the box represents the median; and the ‘x’ represents the mean.

FIG. 5 is a graph showing the rate of change per year in the mPOMA-G score for HPP patients (n=5) prior to treatment with asfotase alfa (pre-treatment) compared to during treatment with asfotase alfa (on-treatment). The P-value based on Wilcoxon rank sum test (exact method) comparing the median mPOMA-G score for pre-treatment and on-treatment is shown. The upper and lower bars represent the maximum and minimum values, respectively; the top and bottom of the box represent the 3rd and 1st quartiles, respectively; the line within the box represents the median; and the ‘x’ represents the mean.

FIG. 6 is a graph showing the mPOMA-G score at baseline and at last assessment for HCs and HPP patients treated with asfotase alfa. Median m-POMA score and patient age with minimum and maximum values are shown.

FIGS. 7A-7B are graphs showing individual changes in mPOMA-G score by age in historical HPP control patients (FIG. 7A) and in HPP patients prior to and following treatment with asfotase alfa (FIG. 7B).

FIGS. 8A-8B are graphs showing individual changes in mPOMA-G score by age from the earliest historical gait data to pre-treatment baseline for patients treated with asfotase alfa (FIG. 8A) and from baseline to last on-treatment assessment (FIG. 8B).

FIG. 9 shows the amino acid sequence of asfotase alfa (STRENSIQ®, Alexion Pharmaceuticals, Inc., SEQ ID NO: 1).

### DETAILED DESCRIPTION

Musculoskeletal defects in HPP can lead to compromised physical function, including impaired mobility. We have discovered that asfotase alfa (SEQ ID NO: 1, STRENSIQ®, Alexion Pharmaceuticals, Inc.) can be used effectively to treat hypophosphatasia (HPP), its symptoms, and decreased physical function associated therewith in patients of about 5 to about 15 years of age for an extended period of time (e.g., at least one year, at least two years, at least three years, at least four years, at least five years, at least six years, at least seven years, at least eight years, at least nine years, at least ten years, or longer than ten years, such as for the lifetime of the patient). As described herein, the Performance-Oriented Mobility Assessment Gait (POMA-G), a clinical gait assessment tool for adults, was modified to accurately assess gait impairment and change in gait impairment in children and adolescents with HPP and was utilized during administration of asfotase alfa treatment.

For example, asfotase alfa can be administered to treat HPP patients of about 5 to about 15 years of age exhibiting gait impairments (e.g., reduced step length, reduced foot clearance, and a widened stance) relative to an untreated HPP subject (e.g., an untreated HPP subject of about the same age, same gender, and/or height). Thus, asfotase alfa is effective for the treatment of HPP and gait impairments associated with HPP in both children (e.g., children having HPP of about 5 to about 12 years of age) and adolescents (e.g., adolescents having HPP of about 12 to about 15 years of age).

Methods for administering asfotase alfa (SEQ ID NO: 1) to an HPP patient of about 5 to about 15 years of age (e.g., a child or an adolescent having HPP) that result in an improvement in gait impairments are described. For example, asfotase alfa can be administered to an HPP patient of about 5 to about 15 years of age having an average modified POMA-G (mPOMA-G) score of about 5 or less (e.g., an mPOMA-G score of 0, 1, 2, 3, or 4). For example, asfotase alfa can be administered to an HPP patient of about 5 to about 15 years of age having an average modified POMA-G (mPOMA-G) score of about 9 or less (e.g., an mPOMA-G score of 6, 7, or 8). Following administration of asfotase alfa, the HPP patient of about 5 to about 15 years of age can exhibit an improvement in one or more mPOMA-G gait assessments, such as gait trunk sway, walking stance, step length and height, step symmetry, and/or step continuity. Accordingly, administration of asfotase alfa can result in an increase in the average mPOMA-G score of the HPP patient of about 5 to about 15 years of age. The average increase is an increase of about 1, 2, 3, 4 or more points in the mPOMA-G, relative to an untreated HPP patient of about the same age, gender, and/or height (e.g., a historical control patient).

Given the results described herein using asfotase alfa, other sALPs (such as a polypeptide variant having at least 95% sequence identity to the sequence of SEQ ID NO: 1) can be used to treat HPP patients of about 5 to about 15 years of age (e.g., a child or an adolescent having HPP) for an extended period of time (e.g., at least one year, at least two years, at least three years, at least four years, at least five years, at least six years, at least seven years, at least eight years, at least nine years, at least ten years, or longer than ten years, such as for the lifetime of the patient). In particular, other sALPs can be used to treat gait impairments in these HPP patients for an extended treatment period.

### Methods of Treatment

Provided herein are methods for treating an HPP patient of about 5 to about 15 years of age, such as a child having HPP (e.g., a child having HPP of about 5 to about 12 years of age) or an adolescent having HPP (e.g., an adolescent having HPP of about 12 to about 15 years of age). HPP patients can be treated by administering an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) across a range of ages, e.g., about 5 to about 7, about 5 to about 8, about 5 to about 9, about 5 to about 10, about 5 to about 11, about 5 to about 12, about 5 to about 13, about 5 to about 14, about 6 to about 8, about 6 to about 9, about 6 to about 10, about 6 to about 11, about 6 to about 12, about 6 to about 13, about 6 to about 14, about 6 to about 15, about 7 to about 9, about 7 to about 10, about 7 to about 11, about 7 to about 12, about 7 to about 13, about 7 to about 14, about 7 to about 15, about 8 to about 10, about 8 to about 11, about 8 to about 12, about 8 to about 13, about 8 to about 14, about 8 to about 15, about 9 to about 11, about 9 to about 12, about 9 to about 13, about 9 to about 14, about 9 to about 15, about 10 to about 12, about 10 to about 13, about 10 to about 14, about 10 to about 15, about 11 to about 13, about 12 to about 14, about 10 to about 15, about 11 to about 13, about 11 to about 14, about 11 to about 15, about 12 to about 14, about 12 to about 15, or about 13 to about 15 years of age.

Patients of about 5 to about 15 years of age (e.g., children and adolescents) can be diagnosed with HPP prior to administration of an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa). An HPP patient of about 5 to about 15 years of age can exhibit, e.g., gait impairments relative to a healthy subject of about the same age and/or gender. Additionally, the HPP patient of about 5 to about 15 years of age may be one that has not previously been treated with an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa).

The method involves administering an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) to an HPP patient of about 5 to about 15 years of age, such as administering an sALP for a period of least one year (e.g., at least two years, at least three years, at least four years, at least five years, at least six years, at least seven years, at least eight years, at least nine years, at least ten years, or longer than ten years, such as for the lifetime of the patient). In particular, an sALP, such as asfotase alfa, can be administered for a period of time to an HPP patient of about 5 to about 15 years of age previously determined to have an average m-POMA-G score of about 8 or less (e.g., about 1, 2, 3, 4, 5, 6, 7, or 8).

After administration of the sALP, the mPOMA-G score of the HPP patient of about 5 to about 15 years of age can be compared to the mPOMA-G score of the patient prior to administration of the sALP or to an untreated subject having HPP. The mPOMA-G of the HPP patient of about 5 to about 15 years of age can also be compared to the mPOMA-G score of a healthy subject (e.g., a subject without HPP). Alternatively, the methods can include determining the mPOMA-G score prior to administering an sALP, such as asfotase alfa.

Additionally, the method can further include performing an additional assessment, such as one or more of a Six Minute Walk Test (6MWT) to assess the walking ability of the HPP patient of about 5 to about 15 years of age, a Child Health Assessment Questionnaire (CHAO), and/or a Pediatric Outcomes Data Collection Instrument (PODCI) to assess the ability of the HPP patient of about 5 to about 15 years of age to perform activities of daily living (ADL) either before or after administration of an sALP. The mPOMA-G can then be used in combination with one or more of the 6MWT, CHAQ, and/or PODCI to assess treatment efficacy using an sALP (such as TNSALP, for example the sALP fusion polypeptide of SEQ ID NO: 1 or a polypeptide variant having at least 95% sequence identity to the sequence of SEQ ID NO: 1, e.g., asfotase alfa), in which improvements relative to a certain value or score demonstrate that the sALP is effective for treating HPP.

For example, when administration of an sALP to an HPP patient of about 5 to about 15 years of age (e.g., a child or adolescent having HPP) results in a yearly increase in the average mPOMA-G score of about 2.5 or more (e.g., about 3, about 3.5, about 4, about 4.5, about 5, about 5.5, about 6, about 6.5, or about 7), in which the patient previously had an average mPOMA-G score of about 9 or less (e.g., about 0, 1, 2, 3, 4, 5, 6, 7, 8, or 9)(baseline), then the sALP treatment is determined to be effective at treating gait impairments associated with HPP. Alternatively, when administration of an sALP does not result in an increase in the average mPOMA-G score of about 2.5 or more, the dosage and/or frequency of sALP administration can be changed (e.g., increased), e.g., until an effective amount of the sALP for the HPP patient of about 5 to about 15 years of age (e.g., a child or adolescent having HPP) is identified. For instance, if necessary, the dosage of the sALP such as TNSALP, for example the sALP fusion polypeptide of SEQ ID NO: 1 or a polypeptide variant having at least 95% sequence identity to the sequence of SEQ ID NO: 1, e.g., asfotase alfa) can be increased from about 6 mg/kg/week to, e.g., about 9 mg/kg/week.

For example, when administration of an sALP to an HPP patient of about 5 to about 15 years of age (e.g., a child or adolescent having HPP) results in an increase in the average mPOMA-G score of about 0.6 or about 1.0 or more (e.g., about 0.7, about 0.8, about 0.9, about 1.0, or about 1.1), in which the patient previously had an average mPOMA-G score of about 9 or less (e.g., about 0, 1, 2, 3, 4, 5, 6, 7, 8, or 9)(baseline), then the sALP treatment is determined to be effective at treating gait impairments associated with HPP. Alternatively, when administration of an sALP does not result in an increase in the average mPOMA-G score of about 0.6 or about 1.0 or more, the dosage and/or frequency of sALP administration can be changed (e.g., increased), e.g., until an effective amount of the sALP for the HPP patient of about 5 to about 15 years of age (e.g., a child or adolescent having HPP) is identified. For instance, if necessary, the dosage of the sALP such as TNSALP, for example the sALP fusion polypeptide of SEQ ID NO: 1 or a polypeptide variant having at least 95% sequence identity to the sequence of SEQ ID NO: 1, e.g., asfotase alfa) can be increased from about 6 mg/kg/week to, e.g., about 9 mg/kg/week.

### Hypophosphatasia in Children and Adolescents

Asfotase alfa is administered, as described herein, to treat childhood HPP or adolescent HPP. In particular, patients with childhood HPP (e.g., children having HPP of about 5 to about 12 years of age) or adolescent HPP (e.g., adolescents with HPP of about 12 to about 18 years of age, such as adolescents with HPP of about 12 to about 15 years of age) can be treated with an sALP (such as TNSALP, for example the sALP fusion polypeptide of SEQ ID NO: 1 or a polypeptide variant having at least 95% sequence identity to the sequence of SEQ ID NO: 1, e.g., asfotase alfa) for a period of at least one year (e.g., at least two years, at least three years, at least four years, at least five years, at least six years, at least seven years, at least eight years, at least nine years, at least ten years, or longer than ten years (e.g., the lifetime of the patient). We observed that an HPP phenotype of, e.g., childhood HPP or adolescent HPP, such as a mobility impairment (e.g., a gait impairment), as assessed using, e.g., an mPOMA-G, improves following treatment with an sALP (such as TNSALP, for example the sALP fusion polypeptide of SEQ ID NO: 1 or a polypeptide variant having at least 95% sequence identity to the sequence of SEQ ID NO: 1, e.g., asfotase alfa).

Accordingly, the methods described herein are useful for alleviating symptoms of HPP described herein, particularly when the sALP (such as TNSALP, for example the sALP fusion polypeptide of SEQ ID NO: 1 or a polypeptide variant having at least 95% sequence identity to the sequence of SEQ ID NO: 1, e.g., asfotase alfa) is administered for a period of at least one year (e.g., at least two years, at least three years, at least four years, at least five years, at least six years, at least seven years, at least eight years, at least nine years, at least ten years, or longer than ten years, such as for the lifetime of the patient). For instance, the methods are useful for treating symptoms of childhood or adolescent HPP, including, but not limited to, gait disturbance; skeletal deformities, such as bowed legs and enlarged wrists, knees, and ankles as a result of flared metaphyses; joint pain; bone pain; bone fracture; muscle weakness; muscle pain; rickets; premature loss of deciduous teeth; incomplete bone mineralization; elevated blood and/or urine levels of phosphoethanolamine (PEA), inorganic pyrophosphate (PPi), or pyridoxal 5′-phosphate (PLP); hypomineralization; rachitic ribs; hypercalciuria; short stature; HPP-related seizure; inadequate weight gain; craniosynostosis; and/or calcium pyrophosphate dihydrate crystal deposition.

The above mentioned symptoms can be treated with an sALP for a period of at least one year (e.g., at least two years, at least three years, at least four years, at least five years, at least six years, at least seven years, at least eight years, at least nine years, at least ten years, or longer than ten years, such as for the lifetime of the patient). The mPOMA-G is a useful metric for evaluating the need for or the efficacy of treatment using an sALP (such as TNSALP, for example the sALP fusion polypeptide of SEQ ID NO: 1 or a polypeptide variant having at least 95% sequence identity to the sequence of SEQ ID NO: 1, e.g., asfotase alfa) in childhood and adolescent HPP.

Modified Performance-Oriented Mobility Assessment—Gait (mPOMA-G)

Children with HPP (e.g., children having HPP of about 5 to about 12 years of age) or adolescents with HPP (e.g., adolescents with HPP of about 12 to about 18 years of age, such as adolescents with HPP of about 12 to about 15 years of age) can be identified for treatment with an sALP (such as TNSALP, for example the sALP fusion polypeptide of SEQ ID NO: 1 or a polypeptide variant having at least 95% sequence identity to the sequence of SEQ ID NO: 1, e.g., asfotase alfa) using a modified version of the gait subtest of the Performance Oriented Mobility Assessment (mPOMA). As is described herein, the mPOMA-G can be used to evaluate gait impairments in HPP patients of about 5 to about 15 years of age (e.g., children or adolescents having HPP) to generate an mPOMA-G score for the patient.

The POMA-G originally included a range of tests to evaluate gait impairments of adults, e.g., elderly and community-dwelling adults, using a 12 point rating scale. The gait subtest of the original POMA-G was described in detail in Tinetti et al. (Am J Med 80:429-434, 1986). A rater, such as a physical therapist, may evaluate HPP patients of about 5 to about 15 years of age, using the POMA-G or mPOMA-G (see, e.g., Tables 2 and 3, respectively, herein below) during a clinical visit or can evaluate patients using video recordings of the patient. The POMA-G provides assessments in the following areas: initiation of gait; step length, height, symmetry, and continuity; foot clearance; path alignment; trunk stability; and walking stance. In each POMA-G area, 0 is the lowest score per item, and 1 or 2 is the highest score per item.

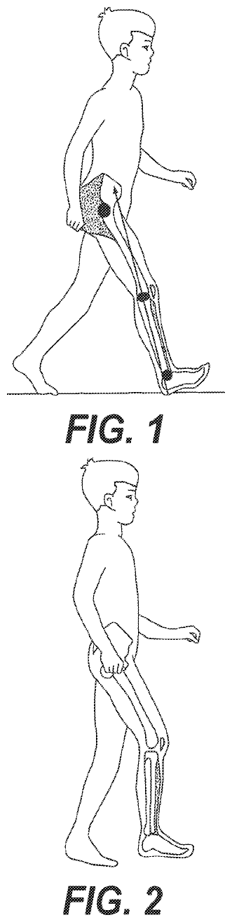

The POMA-G was modified to better assess children and adolescent HPP patients by removing the areas of gait initiation, which can be indicative of neurologic abnormalities instead of mobility impairments, and by removing path, which can be difficult to quantify in children and adolescents, e.g., children and adolescents having HPP. The mPOMA-G was further modified to assess HPP patients of about 5 to about 15 years of age (e.g., children and adolescent HPP patients) in individual areas, such as step length and step continuity, to increase the rating score to a 3 point instead of a 2 point or 1 point scale. In addition, the mPOMA-G can include clarifying descriptions of all items, including trunk sway and walk stance, to increase sensitivity and consistency among raters and/or a scoring key to provide additional details on individual item ratings (see, e.g., Table 3) and examples of patients performing the different areas (see, e.g., FIG. 1 and FIG. 2).

For example, a rater (e.g., a physical therapist) can score an HPP patient of about 5 to about 15 years of age (e.g., a child or adolescent HPP patient) in the mPOMA-G areas of step length and height, step symmetry, step continuity, foot clearance, trunk stability, and walking stance. Step length and height of the HPP patient can be scored by a rater as follows:

a) the right swing foot does not pass the left stance foot with step (0 points), the right heel passes the left stance foot (1 point), or the right foot passes the left stance foot by at least the length of the patient's foot between the stance toe and swing heel (2 points);

b) the right foot does not clear the floor completely with step or the patient raises their foot by more than 1-2 inches (0 points) or the right foot completely clears floor (1 point);

c) the left swing foot does not pass the right stance foot with step (0 points), the left heel passes the right stance foot (1 point), or the left foot passes the right stance foot by at least the length of the patient's foot between the stance toe and the swing heel (2 points); and

d) the left foot does not clear the floor completely with step or the patient raises their foot by more than 1-2 inches (0 points) or the left foot completely clears the floor (1 point).

Step symmetry of the HPP patient of about 5 to about 15 years of age (e.g., a child or adolescent HPP patient) can be scored by a rater based on the observations that the left foot does not clear the floor completely with step or the patient raises their foot by more than 1-2 inches (0 points) or the left foot completely clears the floor (1 point). Stopping or discontinuity between steps of the HPP patient of about 5 to about 15 years of age (e.g., a child or adolescent HPP patient) can be scored by a rater based on the observations that there is stopping or discontinuity between steps (0 points); the steps appear continuous unilaterally (the rater observes the patient raising the heel of one foot as the heel of other foot touches the floor, unilaterally) or there is flat foot contact on the stance limb when the heel of the other foot touches the floor bilaterally with no breaks or stops in stride (1 point); or the steps appear continuous bilaterally (e.g., the rater observes the patient raising the heel of one foot as the heel of other foot touches the floor, bilaterally), with no breaks or stops in stride, and step lengths are equal (2 points).

Trunk stability of the HPP patient of about 5 to about 15 years of age (e.g., a child or adolescent HPP patient) can be scored by a rater based on the observations that there is marked sway (e.g., moderate lateral flexion as the result of instability bilateral or unilateral) or use of walking aids (0 points); no marked sway, but compensatory patterns, such as trunk flexion, knee flexion, arm abduction, or retraction to increase postural stability while walking (1 point); or no sway, no flexion, no use of arms, and no walking aid (2 points).

Walking stance of the HPP patient of about 5 to about 15 years of age (e.g., a child or adolescent HPP patient) can be scored by a rater based on the observations that the heels are always apart and there is a wide base of support utilized to increase postural stability (0 points) or the heels are intermittently apart (1 point).

The individual scores in the mPOMA-G areas of step length and height, step symmetry, step continuity, foot clearance, trunk stability, and walking stance for an HPP patient of about 5 to about 15 years of age (e.g., a child or adolescent HPP patient) can be summed to generate a total mPOMA-G score for the patient ranging from 0 to 12, in which a lower score indicates greater gait impairments and a higher score indicates less or no gait impairments, e.g., gait of a healthy subject (e.g., step length and height points+step symmetry points+step continuity points+foot clearance points+trunk stability points+walking stance points=total mPOMA-G score). The total mPOMA-G score of the HPP patient of about 5 to about 15 years of age (e.g., a child or adolescent HPP patient) can then be compared to the mPOMA-G score of other HPP patients (e.g., a historical control patient) to, e.g., determine the average mPOMA-G score for the HPP patient.

The mPOMA-G score of an HPP patient of about 5 to about 15 years of age (e.g., a child or adolescent HPP patient) will range from about 0 to equal to or less than about 12, in which a higher score (e.g., about 8, 9, 10, 11, or 12) is considered representative of healthy subjects (e.g., subjects without HPP). An HPP patient of about 5 to about 15 years of age (e.g., a child or adolescent HPP patient) with an average mPOMA-G score of less than about 9 (e.g., about 0, 1, 2, 3, 4, 5, 6, 7, 8, or 9) can be treated with an sALP (such as TNSALP, for example the sALP fusion polypeptide of SEQ ID NO: 1 or a polypeptide variant having at least 95% sequence identity to the sequence of SEQ ID NO: 1, e.g., asfotase alfa), such as by administering an sALP for a period of at least one year (e.g., at least two years, at least three years, at least four years, at least five years, at least six years, at least seven years, at least eight years, at least nine years, at least ten years, or longer than ten years, such as for the lifetime of the patient). The mPOMA-G score of the treated patient can then be compared to the mPOMA-G score of an untreated HPP patient, such as an HPP patient of about 5 to about 15 years of age.

The methods can result in an improvement in the mPOMA-G score of an HPP patient of about 5 to about 15 years of age (e.g., a child or adolescent HPP patient). For example, treatment with an sALP (such as TNSALP, for example the sALP fusion polypeptide of SEQ ID NO: 1 or a polypeptide variant having at least 95% sequence identity to the sequence of SEQ ID NO: 1, e.g., asfotase alfa), such as treatment with an sALP for a period of at least one year (e.g., at least two years, at least three years, at least four years, at least five years, at least six years, at least seven years, at least eight years, at least nine years, at least ten years, or longer than ten years, such as for the lifetime of the patient), result in an average yearly increase in the mPOMA-G score of about 2.5 or more (e.g., about 3, about 3.5, about 4, about 4.5, about 5, about 5.5, about 6, about 6.5, or about 7). For example, the mPOMA-G score of the patient of about 5 to about 15 years of age can increase on average to about 7.5 or greater (e.g., to about 8, about 8.5, about 9, about 9.5, about 10, about 10.5, or about 12) after administration of the sALP. Accordingly, administration of the sALP to the patient can result in a decrease in one or more gait impairments, such as reduced step length, reduced foot clearance, and/or widened stance.

The increase in the mPOMA-G can be sustained throughout administration of the sALP (such as TNSALP, for example the sALP fusion polypeptide of SEQ ID NO: 1 or a polypeptide variant having at least 95% sequence identity to the sequence of SEQ ID NO: 1, e.g., asfotase alfa) to the HPP patient of about 5 to about 15 years of age (e.g., a child or adolescent HPP patient), e.g., for a period of at least one year (e.g., at least two years, at least three years, at least four years, at least five years, at least six years, at least seven years, at least eight years, at least nine years, at least ten years, or longer than ten years, such as for the lifetime of the patient). For instance, the mPOMA-G score can increase yearly by about 2.5 points and can remain at ±10% of the increased mPOMA-G score (e.g., about 7.5, about 8, about 8.5, about 9, about 9.5, about 10, about 10.5, or about 12) during treatment with the sALP.

Likewise, the improvement in gait impairment(s) can be sustained throughout administration of the sALP, e.g., for a period of at least one year (e.g., at least two years, at least three years, at least four years, at least five years, at least six years, at least seven years, at least eight years, at least nine years, at least ten years, or longer than ten years, such as for the lifetime of the patient). For instance, the HPP patient exhibits decreased reliance on an assistive device for walking, such as a wheelchair, a wheeled walker, a cane, or an orthotic during treatment with the sALP.

The mPOMA-G score can also be used to assess treatment efficacy of an sALP (such as TNSALP, for example the sALP fusion polypeptide of SEQ ID NO: 1 or a polypeptide variant having at least 95% sequence identity to the sequence of SEQ ID NO: 1, e.g., asfotase alfa), in which improvements relative to a certain mPOMA-G score demonstrate that the sALP is effective for treating gait impairments associated with HPP. For example, when administration of an sALP to an HPP patient of about 5 to about 15 years of age (e.g., a child or adolescent HPP patient) results in an average increase in the mPOMA-G score of about 2.5 or more (e.g., about 3, about 3.5, about 4, about 4.5, about 5, about 5.5, about 6, about 6.5, or about 7), in which the patient previously had an average mPOMA-G score of about 5 or less (e.g., about 0, 1, 2, 3, or 4), then the sALP is considered to be effective at, e.g., treating a gait impairment(s) associated with HPP.

Alternatively, when administration of an sALP for a treatment period of at least one year (e.g., at least two years, at least three years, at least four years, at least five years, at least six years, at least seven years, at least eight years, at least nine years, at least ten years, or longer than ten years, such as for the lifetime of the patient) does not result in an average increase in the mPOMA-G score of about 2.5 or more (e.g., an increase of about 3, about 3.5, about 4, about 4.5, about 5, about 5.5, about 6, about 6.5, or about 7), the dosage and/or frequency of sALP administration can be changed (e.g., increased), e.g., until an effective amount of the sALP for the HPP patient of about 5 to about 15 years of age (e.g., a child or adolescent HPP patient) is identified. For instance, if necessary, the dosage of the sALP (such as TNSALP, for example the sALP fusion polypeptide of SEQ ID NO: 1 or a polypeptide variant having at least 95% sequence identity to the sequence of SEQ ID NO: 1, e.g., asfotase alfa) can be increased from about 6 mg/kg/week to, e.g., about 9 mg/kg/week.

Additionally, the method can further include performing one or more of a 6MWT, CHAQ, and/or PODCI evaluation in combination with the mPOMA-G. The CHAQ or PODCI can be used in combination with the mPOMA-G to assess the ability of the HPP patient of about 5 to about 15 years of age to perform activity of daily living (ADL) after administration of an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa), as is described in U.S. Application No. 62/305,450, hereby incorporated by reference in its entirety. The 6MWT can be used to assess walking ability and physical function of the patient after administration of an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa), as is described in International Application No. PCT/US2016/025721, hereby incorporated by reference in its entirety. The mPOMA-G can then be used in combination with one or more of the 6MWT, CHAQ, and/or PODCI analyses to assess treatment efficacy using an sALP (such as TNSALP, for example the sALP fusion polypeptide of SEQ ID NO: 1 or a polypeptide variant having at least 95% sequence identity to the sequence of SEQ ID NO: 1, e.g., asfotase alfa), in which improvements relative to a certain value or score demonstrate that the sALP is effective for treating HPP.

### Alkaline Phosphatase

Asfotase alfa is a human TNSALP (hTNSALP; SEQ ID NO: 1) fusion polypeptide formulated for the treatment of HPP. In particular, asfotase alfa can be used effectively to treat hypophosphatasia (HPP), its symptoms, and gait impairments associated therewith in HPP patients of about 5 to about 15 years of age (e.g., a child or an adolescent having HPP) for an extended period of time (e.g., at least one year, at least two years, at least three years, at least four years, at least five years, at least six years, at least seven years, at least eight years, at least nine years, at least ten years, or longer than ten years, such as for the lifetime of the patient).

Given the results described herein, the treatment methods are not limited to administration of a particular alkaline phosphatase (ALP) or nucleic acid sequence encoding an ALP. Alkaline phosphatases encompass a group of enzymes that catalyze the cleavage of a phosphate moiety (e.g., hydrolysis of pyrophosphate, PPᵢ). There are four known mammalian alkaline phosphatase (ALP) isozymes: tissue nonspecific alkaline phosphatase (TNSALP; described further below), placental alkaline phosphatase (PLALP; e.g., Accession Nos. P05187, NP_112603, and NP_001623), germ cell alkaline phosphatase (GALP; e.g., Accession No. P10696), and intestinal alkaline phosphatase (IALP; e.g., Accession Nos. P09923 and NP_001622). In addition to the exemplary ALPs discussed above, any polypeptide having the identical or similar catalytic site structure and/or enzymatic activity of ALP can be used (e.g., as an sALP or an sALP fusion polypeptide as defined herein) for treating HPP patients, such as HPP patients of about 5 to about 15 years of age (e.g., a child or an adolescent having HPP). Bone delivery conjugates including sALP are further described in PCT publication Nos. WO 2005/103263 and WO 2008/138131.

TNSALPs that can be used according to the methods described herein include, e.g., human TNSALP (Accession Nos. NP_000469, AAI10910, AAH90861, AAH66116, AAH21289, and AA126166); rhesus TNSALP (Accession No. XP_01109717); rat TNSALP (Accession No. NP_037191); dog TNSALP (Accession No. AAF64516); pig TNSALP (Accession No. AAN64273); mouse (Accession No. NP 031457); cow TNSALP (Accession Nos. NP_789828, NP_776412, AAM 8209, and AAC33858); cat TNSALP (Accession No. NP_001036028); and variants thereof having 90, 95, 97, or 99% sequence identity to any one of SEQ ID NOs: 7-19. In particular, TNSALP can be a recombinant human TNSALP (e.g., SEQ ID NO: 1, asfotase alfa; see U.S. Pat. Nos. 7,763,712 and 7,960,529, incorporated herein by reference in their entirety) used for the treatment of HPP patients of about 5 to about 15 years of age (e.g., a child or an adolescent having HPP). The TNSALP can also be one that exhibits at least about 95% sequence identity to the polypeptide or nucleic acid sequence of the above-noted TNSALPs.

### Soluble Alkaline Phosphatases

The ALPs that can be used in the methods include soluble (e.g., extracellular or non-membrane-bound) forms of any of the alkaline phosphatases described herein. The sALP can be, for example, a soluble form of human tissue non-specific alkaline phosphatase (human TNSALP (hTNSALP)). The methods are not limited to a particular sALP and can include any sALP that is physiologically active toward, e.g., phosphoethanolamine (PEA), inorganic pyrophosphate (PPi), and pyridoxal 5′-phosphate (PLP). In particular, an sALP is one that is catalytically competent to improve skeletal mineralization in bone. The methods further include nucleic acids encoding the sALPs described herein that can be used to treat HPP patients of about 5 to about 15 years of age (e.g., a child or an adolescent having HPP).

TNSALP is a membrane-bound protein anchored by a glycolipid moiety at the C-terminal (Swiss-Prot, P05186). This glycolipid anchor (GPI) is added post-translationally after the removal of a hydrophobic C-terminal end, which serves both as a temporary membrane anchor and as a signal for the addition of the GPI. While the GPI anchor is located in the cell membrane, the remaining portions of TNSALP are extracellular. In particular, TNSALP (e.g., human TNSALP (hTNSALP)) can be engineered to replace the first amino acid of the hydrophobic C-terminal sequence (an alanine) with a stop codon, thereby producing an engineered hTNSALP that contains all amino acid residues of the native anchored form of TNSALP and lacks the GPI membrane anchor. One skilled in the art will appreciate that the position of the GPI membrane anchor will vary in different ALPs and can include, e.g., the last 10, 12, 14, 16, 18, 20, 21, 22, 23, 24, 25, 26, 27, 28, 29, 30, 32, 34, 36, 38, 40, 45, 50, or more amino acid residues on the C-terminus of the polypeptide. Recombinant sTNSALP can include, e.g., amino acids 1 to 502 (18 to 502 when secreted), amino acids 1 to 501 (18 to 501 when secreted), amino acids 1 to 504 (18 to 504 when secreted), amino acids 1 to 505 (18-505 when secreted), or amino acids 1 to 502. Thus, the C-terminal end of the native ALP can be truncated by certain amino acids without affecting ALP activity.

In addition to the C-terminal GPI anchor, TNSALP also has an N-terminal signal peptide sequence. The N-terminal signal peptide is present on the synthesized protein when it is synthesized, but cleaved from TNSALP after translocation into the ER. The sALPs include both secreted (i.e., lacking the N-terminal signal) and non-secreted (i.e., having the N-terminal signal) forms thereof. One skilled in the art will appreciate that the position of the N-terminal signal peptide will vary in different alkaline phosphatases and can include, for example, the first 5, 8, 10, 11, 12, 13, 14, 15, 16, 17, 18, 19, 20, 21, 22, 23, 24, 25, 27, 30, or more amino acid residues on the N-terminus of the polypeptide. One of skill in the art can predict the position of a signal sequence cleavage site, e.g., by an appropriate computer algorithm such as that described in Bendtsen et al. (J. Mol. Biol. 340(4):783-795, 2004) and/or at www.cbs.dtu.dk/services/SignalP/.

The methods can also be performed using sALP consensus sequences derived from the extracellular domain of ALP isozymes (e.g., TNSALP, PALP, GCALP, or IALP). Thus, similar to sTNSALP discussed above, the present disclosure also provides other soluble human ALP isozymes, i.e., without the peptide signal, preferably comprising the extracellular domain of the ALPs. The sALPs also include polypeptide sequences satisfying a consensus sequence derived from the ALP extracellular domain of human ALP isozymes and of mammalian TNSALP orthologs (human, mouse, rat, cow, cat, and dog) or a consensus derived from the ALP extracellular domain of just mammalian TNSALP orthologs (human, mouse, rat, cow, cat, and dog). The sALPs also include those which satisfy similar consensus sequences derived from various combinations of these TNSALP orthologs or human ALP isozymes. Such consensus sequences are given, for example, in WO 2008/138131.

sALPs of the present methods can include not only the wild-type sequence of the sALPs described above, but any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to these alkaline phosphatases (e.g., SEQ ID NOs: 1-24; for example the sALP fusion polypeptide of SEQ ID NO: 1 or a polypeptide variant having at least 95% sequence identity to the sequence of SEQ ID NO: 1, e.g., asfotase alfa). Examples of mutations that can be introduced into an ALP sequence are described in US Patent Application Publication No. 2013/0323244, hereby incorporated by reference in its entirety. An sALP can optionally be glycosylated at any appropriate one or more amino acid residues. In addition, an sALP can have at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to any of the sALPs described herein (such as TNSALP, for example the sALP fusion polypeptide of SEQ ID NO: 1 or a polypeptide variant having at least 95% sequence identity to the sequence of SEQ ID NO: 1, e.g., asfotase alfa). An sALP can have 1, 2, 3, 4, 5, 6, 7, 8, 9, 10, or more additions, deletions, or substitutions relative to any of the sALPs described herein (such as TNSALP, for example the sALP fusion polypeptide of SEQ ID NO: 1 or a polypeptide variant having at least 95% sequence identity to the sequence of SEQ ID NO: 1, e.g., asfotase alfa).

sALP Fusion Polypeptides

Any of the sALPs (such as TNSALP, for example the sALP fusion polypeptide of SEQ ID NO: 1 or a polypeptide variant having at least 95% sequence identity to the sequence of SEQ ID NO: 1, e.g., asfotase alfa), linkers, spacers (e.g., Fc regions), and bone-targeting moieties described herein can be combined in a fusion polypeptide, which includes the structures Z-sALP-Y-spacer-X-Wₙ-V, Z-Wn-X-spacer-Y-sALP-V, Z-sALP-Y-Wn-X-spacer-V, and Z-Wn-X-sALP-Y-spacer-V. In particular, the structure of the sALP fusion polypeptide can be Z-sALP-Y-spacer-X-Wn-V or Z-Wn-X-spacer-Y-sALP-V. The sALP of the sALP fusion polypeptide can be the full-length ALP or functional fragments of ALPs, such as the soluble, extracellular domain of the ALP, as is described herein (e.g., TNSALP, PALP, GCALP and IALP).

Any one of X, Y, Z, and V and/or the spacer can be absent or a linker region including an amino acid sequence of at least one amino acid. For example, X, Y, Z, and V may be a dipeptide sequence (e.g., leucine-lysine or aspartic acid-isoleucine), such as a two residue linker at the Y position (e.g., leucine-lysine) or a two residue linker at the X position (e.g., aspartic acid-isoleucine). For example, sALP fusion polypeptides can have the structure hTNSALP-Fc-D₁₀ (e.g., an sALP fusion polypeptide including the amino acid sequence of SEQ ID NO: 1 or a polypeptide variant having at least 95% sequence identity to the sequence of SEQ ID NO: 1, e.g., asfotase alfa).

The linker region can be of any sequence and length that allows the sALP to remain biologically active, e.g., not sterically hindered. Exemplary linker lengths are between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. For instance, linkers include or consist of flexible portions, e.g., regions without significant fixed secondary or tertiary structure. Exemplary flexible linkers are glycine-rich linkers, e.g., containing at least 50%, 60%, 65%, 70%, 75%, 80%, 85%, 90%, 95%, or even 100% glycine residues. Linkers can also contain, e.g., serine residues. In some cases, the amino acid sequence of linkers consists only of glycine and serine residues. A linker can optionally be glycosylated at any appropriate one or more amino acid residues. Additionally, a linker as described herein can include any other sequence or moiety, attached covalently or non-covalently. The linker can also be absent, in which the spacer (e.g., the Fc region) and the sALP are fused together directly, with no intervening residues.

Useful spacers include, but are not limited to, polypeptides including a Fc region. For example, an sALP can be a fusion polypeptide including an Fc region of an immunoglobulin at the N-terminal or C-terminal domain. An immunoglobulin molecule has a structure that is well known in the art. It includes two light chains (˜23 kD each) and two heavy chains (˜50-70 kD each) joined by inter-chain disulfide bonds. Immunoglobulins are readily cleaved proteolytically (e.g., by papain cleavage) into Fab (containing the light chain and the VH and CH1 domains of the heavy chain) and Fc (containing the CH2 and CH3 domains of the heavy chain, along with adjoining sequences) fragments. Useful Fc fragments as described herein include the Fc fragment of any immunoglobulin molecule, including IgG, IgM, IgA, IgD, or IgE, and their various subclasses (e.g., IgG-1, IgG-2, IgG-3, IgG-4, IgA-1, IgA-2), from any mammal (e.g., human). For instance, the Fc fragment is human IgG-1. The Fc fragments can include, for example, the CH2 and CH3 domains of the heavy chain and any portion of the hinge region. The Fc region can optionally be glycosylated at any appropriate one or more amino acid residues known to those skilled in the art. In particular, the Fc fragment of the fusion polypeptide has the amino acid sequence of SEQ ID NO: 20, or has at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 20. Engineered, e.g., non-naturally occurring, Fc regions can be incorporated into the sALP fusion polypeptides described herein, e.g., those described in International Application Pub. No. W02005/007809, which is hereby incorporated by reference. An Fc fragment as described herein can have 1, 2, 3, 4, 5, 6, 7, 8, 9, 10, 11, 12, 13, 14, 15, 16, 17, 18, 19, 20, 25, 30, 35, 40, 50, or more additions, deletions, or substitutions relative to any of the Fc fragments described herein.

Wₙ can be a bone-targeting moiety, e.g., having a series of consecutive aspartate (D) or glutamate (E) residues, in which n=1 to 50, e.g., n=3-30, e.g., 5-15, e.g., 1, 2, 3, 4, 5, 6, 7, 8, 9, 10, 11, 12, 13, 14, 15, 16, 17, 18, 19, 20, 21, 22 , 23, 24, 25, 26, 27, 28, 29, 30, 31, 32, 33, 34, 35, 36, 36, 38, 39, 40, 41, 42, 43, 44, 45, 46, 47, 48, 49, or 50. The bone-targeting moiety, if present, can be positioned anywhere in the fusion polypeptide, e.g., at or near the N-terminal or C-terminal end, and/or in the linker region. For instance, the bone-targeting moiety can be present at the C-terminal end of an sALP fusion polypeptide. sALP fusion polypeptides can also lack a bone-targeting moiety.

Additional amino acid residues can be introduced into the polypeptide according to the cloning strategy used to produce the fusion polypeptides. For instance, the additional amino acid residues do not provide an additional GPI anchoring signal so as to maintain the polypeptide in a soluble form. Furthermore, any such additional amino acid residues, when incorporated into the polypeptide of the methods, do not provide a cleavage site for endoproteases of the host cell. The likelihood that a designed sequence would be cleaved by the endoproteases of the host cell can be predicted as described, e.g., by Ikezawa (Biol. Pharm. Bull. 25:409-417, 2002).

The sALP fusion polypeptides (such as a TNSALP, for example the sALP fusion polypeptide of SEQ ID NO: 1 or a polypeptide variant having at least 95% sequence identity to the sequence of SEQ ID NO: 1, e.g., asfotase alfa) can be associated into dimers or tetramers. For example, two sALP-Fc monomers can covalently be linked through two disulfide bonds located in the hinge regions of the Fc fragments. Additionally, the sALP fusion polypeptide (e.g., an sALP or an sALP fusion polypeptide) can be glycosylated or PEGylated.

### Production of Nucleic Acids and Polypeptides

The nucleic acids encoding sALP (such as TNSALP, for example the sALP fusion polypeptide of SEQ ID NO: 1 or a polypeptide variant having at least 95% sequence identity to the sequence of SEQ ID NO: 1, e.g., asfotase alfa) can be produced by any method known in the art. Typically, a nucleic acid encoding the desired polypeptide is generated using molecular cloning methods, and is generally placed within a vector, such as a plasmid or virus. The vector is used to transform the nucleic acid into a host cell appropriate for the expression of the fusion polypeptide. Representative methods are disclosed, for example, in Maniatis et al. (Cold Springs Harbor Laboratory, 1989). Many cell types can be used as appropriate host cells, although mammalian cells are preferable because they are able to confer appropriate post-translational modifications. Host cells can include, e.g., Chinese Hamster Ovary (CHO) cell, L cell, C127 cell, 3T3 cell, BHK cell, COS-7 cell or any other suitable host cell known in the art. For example, the host cell is a Chinese Hamster Ovary (CHO) cell (e.g., a CHO-DG44 cell).

The sALPs (such as TNSALP, for example the sALP fusion polypeptide of SEQ ID NO: 1 or a polypeptide variant having at least 95% sequence identity to the sequence of SEQ ID NO: 1, e.g., asfotase alfa) can be produced under any conditions suitable to effect expression of the sALP polypeptide in the host cell. Such conditions include appropriate selection of a media prepared with components such as a buffer, bicarbonate and/or HEPES, ions like chloride, phosphate, calcium, sodium, potassium, magnesium, iron, carbon sources like simple sugars, amino acids, potentially lipids, nucleotides, vitamins and growth factors like insulin; regular commercially available media like alpha-MEM, DMEM, Ham's-F12, and IMDM supplemented with 2-4 mM L-glutamine and 5% Fetal bovine serum; regular commercially available animal protein free media (i.e., HYCLONETM, GE Healthcare; SFM4CHO, Sigma CHO DHFR⁻; Cambrex POWERTM CHO CD supplemented with 2-4 mM L-glutamine, etc.). These media are desirably prepared without thymidine, hypoxanthine and L-glycine to maintain selective pressure, allowing stable protein-product expression.

### Pharmaceutical Compositions and Formulations

A composition including an sALP that can be used in the methods (such as TNSALP, for example the sALP polypeptide of SEQ ID NO: 1 or a polypeptide variant having at least 95% sequence identity to the sequence of SEQ ID NO: 1, e.g., asfotase alfa) can be administered by a variety of methods known in the art. For example, asfotase alfa (SEQ ID NO: 1) can be administered at a range of dosages, in a variety of formulations, and in combination with pharmaceutically acceptable carriers or vehicles. In particular, asfotase alfa is a sterile, preservative-free, nonpyrogenic, clear, slightly opalescent or opalescent, colorless to slightly yellow, with few small translucent or white particles, aqueous solution that is formulated for, e.g., subcutaneous administration. Asfotase alfa can be supplied in glass single-use vials containing asfotase alfa in combination with dibasic sodium phosphate, heptahydrate; monobasic sodium phosphate, monohydrate; and sodium chloride at a pH between 7.2 and 7.6.

Dosage

Any amount of a pharmaceutical composition including an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be administered to HPP patients of about 5 to about 15 years of age (e.g., a child or an adolescent having HPP). The dosages will depend on many factors including the mode of administration and the age of the patient. Typically, the amount of the composition including an sALP contained within a single dose will be an amount that is effective to treat HPP as described herein without inducing significant toxicity.

For example, the sALPs (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be administered to HPP patients of about 5 to about 15 years of age (e.g., a child or an adolescent having HPP, in particular, those having an mPOMA-G score of less than about 8 (e.g., less than, or about, 5)), in individual doses ranging, e.g., from 0.01 mg/kg to 500 mg/kg (e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, 0.5 mg/kg to 25 mg/kg, 1.0 mg/kg to 10 mg/kg, 1.5 mg/kg to 5 mg/kg, or 2.0 mg/kg to 3.0 mg/kg) or from 1 pg/kg to 1,000 μg/kg (e.g., from 5 μg/kg to 1,000 μg/kg, from 1 μg/kg to 750 μg/kg, from 5 μg/kg to 750 pg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 μg/kg).

Exemplary doses of an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) include, but are not limited to, 0.01, 0.05, 0.1, 0.5, 1, 2, 2.5, 5, 10, 20, 25, 50, 100, 125, 150, 200, 250, or 500 mg/kg; or 1, 2, 2.5, 5, 10, 20, 25, 50, 100, 125, 150, 200, 250, 500, 750, 900, or 1,000 μg/kg. In particular, compositions including sALP in accordance with the present disclosure can be administered to HPP patients (e.g., children and adolescents with HPP) in doses ranging from about 0.001 mg/kg/day to about 500 mg/kg/day, about 0.01 mg/kg/day to about 100 mg/kg/day, or about 0.01 mg/kg/day to about 20 mg/kg/day.

For example, the sALP compositions (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be administered to patients in a weekly dosage ranging, e.g., from about 0.5 mg/kg/week to about 140 mg/kg/week, e.g., about 0.8 mg/kg/week to about 50 mg/kg/week, or about 1 mg/kg/week to about 10 mg/kg/week (e.g., about 6 or about 9 mg/kg/week). In particular, the sALP (such as TNSALP, e.g., asfotase alfa) can be administered at a dosage of 2 mg/kg three times a week (total dose 6 mg/kg/week), 1 mg/kg six times a week (total dose 6 mg/kg/week), 3 mg/kg three times a week (total dose 9 mg/kg/week), 0.5 mg/kg three times a week (total dose of 1.5 mg/kg/week), or 9.3 mg/kg three times a week (total dose 28 mg/kg/week). The dosage will be adapted by the clinician in accordance with conventional factors, such as the extent of the disease, and different parameters from the HPP patient, such as an HPP patient of about 5 to about 15 years of age (e.g., a child or an adolescent having HPP), in particular, those having an mPOMA-G score of less than about 8 (e.g., less than, or about, 5).

Dosages of compositions including sALPs (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be provided in either a single or multiple dosage regimens. Doses can be administered, e.g., hourly, bihourly, daily, bidaily, twice a week, three times a week, four times a week, five times a week, six times a week, weekly, biweekly, monthly, bimonthly, or yearly. Alternatively, doses can be administered, e.g., twice, three times, four times, five times, six times, seven times, eight times, nine times, 10 times, 11 times, or 12 times per day. In particular, the dosing regimen is once weekly. The duration of the dosing regimen can be, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s), week(s), or month(s), or even for the remaining lifespan of the HPP patient of about 5 to about 15 years of age (e.g., a child or an adolescent having HPP).

An sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be formulated as a solution for injection, which is a clear, colorless to slightly yellow, aqueous solution, pH 7.4. The sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) may be formulated at a concentration of 12mg/0.3mL,18mg/0.45mL, 28mg/0.7mL, 40mg/1m1, or 80mg/0.8mL.

For example, the composition can be formulated as a 40 mg/ml solution for injection, in which each ml of solution contains 40 mg of sALP (e.g., each vial contains 0.3 ml solution and 12 mg of sALP (40 mg/ml), each vial contains 0.45 ml solution and 18 mg of sALP (40 mg/ml), each vial contains 0.7 ml solution and 28 mg of sALP (40 mg/ml), or each vial contains 1.0 ml solution and 40 mg of asfotase alfa (40 mg/mI)). Additionally, an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be formulated as a solution for injection at a concentration of 100 mg/ml, in which each 1 ml of solution contains 100 mg of sALP (e.g., each vial contains 0.8 ml solution and 80 mg of asfotase alfa (100 mg/mI)).

For example, the recommended dosage of an sALP (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) is 2 mg/kg of body weight administered subcutaneously three times per week, or a dosage regimen of 1 mg/kg of body weight administered subcutaneously six times per week. Additional dosage information is provided below (Table 1).

TABLE 1
DOSING OF ASFOTASE ALFA

| | If injecting 3× per week | | | | If injecting 6× per week | | | | |
|---|---|---|---|---|---|---|---|---|---|
| Body | Dose | Volume | Vial type | | Dose | | Volume | Vial type | |
| Weight | to be | to be | used for | | to be | | to be | used for | |
| (kg) | injected | injected | injection | | injected | | injected | injection | |
| 3 | 6 mg | 0.15 ml | 0.3 | ml | | | | | |
| 4 | 8 mg | 0.20 ml | 0.3 | ml | | | | | |
| 5 | 10 mg | 0.25 ml | 0.3 | ml | | | | | |
| 6 | 12 mg | 0.30 ml | 0.3 | ml | 6 | mg | 0.15 ml | 0.3 | ml |
| 7 | 14 mg | 0.35 ml | 0.45 | ml | 7 | mg | 0.18 ml | 0.3 | ml |
| 8 | 16 mg | 0.40 ml | 0.45 | ml | 8 | mg | 0.20 ml | 0.3 | ml |
| 9 | 18 mg | 0.45 ml | 0.45 | ml | 9 | mg | 0.23 ml | 0.3 | ml |
| 10 | 20 mg | 0.50 ml | 0.7 | ml | 10 | mg | 0.25 ml | 0.3 | ml |
| 11 | 22 mg | 0.55 ml | 0.7 | ml | 11 | mg | 0.28 ml | 0.3 | ml |
| 12 | 24 mg | 0.60 ml | 0.7 | ml | 12 | mg | 0.30 ml | 0.3 | ml |
| 13 | 26 mg | 0.65 ml | 0.7 | ml | 13 | mg | 0.33 ml | 0.45 | ml |
| 14 | 28 mg | 0.70 ml | 0.7 | ml | 14 | mg | 0.35 ml | 0.45 | ml |
| 15 | 30 mg | 0.75 ml | 1 | ml | 15 | mg | 0.38 ml | 0.45 | ml |
| 16 | 32 mg | 0.80 ml | 1 | ml | 16 | mg | 0.40 ml | 0.45 | ml |
| 17 | 34 mg | 0.85 ml | 1 | ml | 17 | mg | 0.43 ml | 0.45 | ml |
| 18 | 36 mg | 0.90 ml | 1 | ml | 18 | mg | 0.45 ml | 0.45 | ml |
| 19 | 38 mg | 0.95 ml | 1 | ml | 19 | mg | 0.48 ml | 0.7 | ml |
| 20 | 40 mg | 1.00 ml | 1 | ml | 20 | mg | 0.50 ml | 0.7 | ml |
| 25 | 50 mg | 0.50 ml | 0.8 | ml | 25 | mg | 0.63 ml | 0.7 | ml |
| 30 | 60 mg | 0.60 ml | 0.8 | ml | 30 | mg | 0.75 ml | 1 | ml |
| 35 | 70 mg | 0.70 ml | 0.8 | ml | 35 | mg | 0.88 ml | 1 | ml |
| 40 | 80 mg | 0.80 ml | 0.8 | ml | 40 | mg | 1.00 ml | 1 | ml |

| | | | | | |
|---|---|---|---|---|---|
| 50 | 50 | mg | 0.50 ml | 0.8 | ml |
| 60 | 60 | mg | 0.60 ml | 0.8 | ml |
| 70 | 70 | mg | 0.70 ml | 0.8 | ml |
| 80 | 80 | mg | 0.80 ml | 0.8 | ml |

| | | | | |
|---|---|---|---|---|
| 90 | 90 | mg | 0.90 ml | 0.8 ml |
| | | | | (×2) |
| 100 | 100 | mg | 1.00 ml | 0.8 ml |
| | | | | (×2) |

Formulations

A composition including sALPs (such as TNSALP, for example the sALP polypeptide of SEQ ID NO: 1 or a polypeptide variant having at least 95% sequence identity to the sequence of SEQ ID NO: 1, e.g., asfotase alfa) can be formulated according to standard methods. Pharmaceutical formulation is a well-established art, and is further described in, e.g., Gennaro (2000) Remington: The Science and Practice of Pharmacy, 20^{th} Edition, Lippincott, Williams & Wilkins (ISBN: 0683306472); Ansel et al. (1999) Pharmaceutical Dosage Forms and Drug Delivery Systems, 7th Edition, Lippincott Williams & Wilkins Publishers (ISBN: 0683305727); and Kibbe (2000) Handbook of Pharmaceutical Excipients American Pharmaceutical Association, 3^{rd} Edition (ISBN: 091733096X). For instance, an sALP composition (such as TNSALP, for example the sALP polypeptide of SEQ ID NO: 1 or a polypeptide variant having at least 95% sequence identity to the sequence of SEQ ID NO: 1, e.g., asfotase alfa) can be formulated as a buffered solution at a suitable concentration and suitable for storage at about 2-8° C. (e.g., about 4° C.). A composition can also be formulated for storage at a temperature below about 0° C. (e.g., about −20° C. or about −80° C.). A composition can further be formulated for storage for up to about 2 years (e.g., one month, two months, three months, four months, five months, six months, seven months, eight months, nine months, 10 months, 11 months, 1 year, 1½ years, or 2 years) at about 2-8° C. (e.g., about 4° C.). Thus, the compositions described herein can be stable in storage for at least about 1 year at about 2-8° C. (e.g., about 4° C.).

The compositions including sALPs (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application.

For example, compositions intended for systemic or local delivery can be in the form of injectable or infusible solutions. Accordingly, the sALP compositions (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) can be formulated for administration by a parenteral mode (e.g., subcutaneous, intravenous, intraperitoneal, or intramuscular injection).

The sALP compositions (such as TNSALP, for example the sALP fusion polypeptide of SEQ ID NO: 1 or a polypeptide variant having at least 95% sequence identity to the sequence of SEQ ID NO: 1, e.g., asfotase alfa) can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The sALP compositions (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) described herein can also be formulated in immunoliposome compositions. Such formulations can be prepared by methods known in the art, such the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

Compositions including an sALP (such as TNSALP, for example the sALP polypeptide of SEQ ID NO: 1 or a polypeptide variant having at least 95% sequence identity to the sequence of SEQ ID NO: 1, e.g., asfotase alfa) can also be formulated with a carrier that will protect the sALP composition against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J.R. Robinson (1978) Sustained and Controlled Release Drug Delivery Systems, Marcel Dekker, Inc., New York.

When sALP compositions (such as TNSALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times.

Carriers/vehicles

Preparations containing an sALP (such as TNSALP, for example the sALP polypeptide of SEQ ID NO: 1 or a polypeptide variant having at least 95% sequence identity to the sequence of SEQ ID NO: 1, e.g., asfotase alfa) can be provided to an HPP patient of about 5 to about 15 years of age, such as a child or an adolescent having HPP, in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. For example, the pharmaceutically acceptable carrier can include sodium chloride and/or sodium phosphate, in which the composition includes, e.g., about 150 mM sodium chloride and/or about 25 mM sodium phosphate, pH 7.4.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

The following examples are intended to illustrate, rather than limit, the disclosure. These studies feature the administration of asfotase alfa (SEQ ID NO: 1) to a patient of about 5 to about 15 years of age, such as a child or an adolescent, to treat HPP, its symptoms, and gait impairments associated therewith for an extended period of time.

### Example 1

### Modification of Performance-Oriented Mobility Assessment—Gait (modified POMA-G, mPOMA-G) to Evaluate Gait in HPP Patients

An analysis of gait was performed to compare children and adolescent HPP patients treated with asfotase alfa (SEQ ID NO: 1) to children and adolescent HPP patients that were not treated with asfotase alfa (the historical HPP control (“HC”)). Gait impairment has a high prevalence as measured at baseline in asfotase alfa clinical trials, i.e., 100% (8/8) in ages 5-12 and 83% (10/12) in ages≥13. Gait performance was assessed using a version of the 12-point Performance-Oriented Mobility Assessment—Gait (POMA-G) which was modified in order to provide improved sensitivity for HPP-related impairments (the modified POMA-G or mPOMA-G). The POMA-G had been used previously to evaluate function in adult populations, including patients with amyotrophic lateral sclerosis, normal pressure hydrocephalus, Parkinson's disease, and stroke, and had been validated in elderly and community-dwelling adults (Tinetti, M.E., et al., Am J Med 80(3):429-34 (1986); Faber, M.J., et al., Arch Phys Med Rehabil. 87(7):885-96 (2006); Means K.M., et al., Arch Phys Med Rehabil. 79(12):1570-6 (1998). The POMA-G has been also been used in deaf children to evaluate balance and gait (Melo et al. Rev. Paul. Pediatr. 30(3): 385-91, 2012). Components of the POMA-G can be used to directly or indirectly measure performance deficits exhibited in HPP patients of about 5 to about 15 years of age; however, the POMA-G, without these modifications, was less appropriate for adequate assessment of HPP patients of about 5 to about 15 years of age.

In particular, the POMA-G can be used to measure step length and height, step symmetry and continuity, trunk sway, and stance, all of which may be abnormal in HPP patients. The POMA-G includes assessments of initiation of gait, step length and step clearance height, symmetry, and continuity; path deviation; trunk sway; and walking stance (Table 2). The lowest score is 0 per item, while 1 or 2 is the highest score per item. Individual item scores are summed to generate a 0 to 12 scale of disability, with lower scores indicating more significant gait impairments and higher scores indicating fewer gait impairments, which would be indicative of, e.g., the gait of a healthy subject.

TABLE 2
Unmodified Performance Oriented Mobility
Assessment - Gait (POMA-G) Assessments

| Assessment | | Observation | Score |
|---|---|---|---|
| 1 | Initiation of gait | Any hesitancy or multiple attempts | =0 |
| | | No hesitancy | =1 |
| 2 | Step length and height | | |
| | a) Right swing foot | Does not pass the left stance foot with step | =0 |
| | | Passes the left stance foot | =1 |
| | b) Right foot clear | Does not clear floor completely with step | =0 |
| | | Right foot completely clears floor | =1 |
| | c) Left swing foot | Does not pass the right stance foot with step | =0 |
| | | Passes the right stance foot | =1 |
| | d) Left foot clear | Does not clear floor completely with step | =0 |
| | | Left foot completely clears floor | =1 |
| 3 | Step symmetry | Right and left step length not equal | =0 |
| | | Left foot completely clears floor | =1 |
| 4 | Step continuity | Stopping or discontinuity between steps | =0 |
| | | Steps appear continuous | =1 |
| 5 | Path | Marked deviation | =0 |
| | | Mild/moderate deviation or uses walking aid | =1 |
| | | Straight without walking aid | =2 |
| 6 | Trunk | Marked sway or uses walking aid | =0 |
| | | No sway but flexion of knees or back or spreads | =1 |
| | | arms out while walking | =2 |
| | | No sway, no flexion, no use of arms, and no | |
| | | walking aid | |
| 7 | Walk stance | Heels apart | =0 |
| | | Heels almost touching while walking | =1 |

| | |
|---|---|
| Gait score | /12 |

The validity of each component of the POMA-G as a measure of clinically important musculoskeletal defects in HPP patients was confirmed by evaluating each component of the scale in the context of physical disabilities specific to HPP. The measures most relevant to pathology in patients with HPP are those that reflect proximal and lower extremity muscle weakness, stability, and base of support. A total of six assessments in the POMA-G specifically measure criteria that indicate proximal and lower extremity muscle weakness, stability, and/or base of support. The specific features of the POMA-G that are relevant to HPP are described below.
Trunk sway and walking stance (wide base of support to increase postural stability) are indicators of proximal muscle weakness. Abnormalities in these parameters are associated with difficulty navigating obstacles, poor balance, and increased risk of collisions or falls.
Step length and height are important indicators of both proximal and lower extremity strength. Abnormalities in these parameters interfere with the ability to navigate irregular surfaces and increase the risk of falls and injury.
Gait asymmetry reflects structural discrepancies between limbs commonly seen in HPP. Gait asymmetry is an important functional measure, because it compromises the ability to navigate obstacles and increases the risk of collisions and falls.
Gait continuity reflects balance, stability, and fluidity of movement. Gait continuity can affect the patient safety, as forward movement is delayed when step continuity is not present, affecting the stability of gait and risk of fall.

On the other hand, gait initiation is indicative of neurologic abnormality (e.g., Parkinson's disease) and is less likely to be secondary to musculoskeletal defects; therefore, gait initiation was not expected to be affected by the physical disabilities specific to HPP. Additionally, potential issues identified for the item “path” included a possible lack of focused and purposeful gait in children and the potential difficulty in quantifying path deviation in the historical videos in the absence of specific floor markers. The following HPP-specific modifications of the POMA-G were made to optimize the discriminant value of the assessment in patients with HPP, while preserving the clinometric properties of the underlying instrument.
Item 1 (initiation of gait) was removed, because patients with HPP do not typically have difficulty initiating gait.
Item 5 (path) was removed, because the nature and quality of the clinical video footage was not expected to permit a reliable assessment of path deviations, as well as the potential for the absence of focused and purposeful movement as typical of young children.
Item 2 (step length), the scale was changed from a 2 point (0, 1) rating scale to a 3 point (0, 1, 2) scale with detailed definitions for each score to provide greater sensitivity and precision to detect change. Clinical relevance applied to increased risk of trips and falls, decreased ability to climb standard stairs or to climb school bus stairs with an increased vertical rise. Similar to ambulation, stair climbing with a mature pattern of alternating feet requires adequate step length and hip extension on the stance limb. A reduced step size requires more steps to ambulate a given distance, a possible increased fatigue and difficulties keeping up with peers.
Item 4 (step continuity), the scale was changed from a 2 point (0, 1) rating scale to a 3 point (0, 1, 2) scale to have a partial credit score of 2 for emerging step continuity and greater sensitivity and precision to detect change. A score of two was given if step continuity was not seen bilaterally and was only present on one side or if bilaterally a heel strike was present at initial contact with a foot flat position on the stance limb. Clinical relevance applied to slow and inefficient ambulation and reduced stability that could impact ability to move between classes in the allotted time and difficulties with participation in school activities.

In addition, the resulting mPOMA-G analysis included clarifying descriptions of all items, including trunk sway and walk stance, to increase sensitivity and consistency among raters were provided. A scoring key was also created to provide additional detail on item rating and to include examples of a child demonstrating step continuity (FIG. 1) and for step clearance, evaluation of hip and knee flexion at mid-swing (FIG. 2) for reference during rating.

The mPOMA-G, optimized for analysis of HPP patients, is shown below (Table 3). Similarly to the POMA-G, 0 is the lowest score per item, and 1 or 2 is the highest score per item. As in the POMA-G, individual item scores are summed to generate a 0 to 12 scale of disability, with lower scores indicating greater disability. The modifications improved the HPP-specific sensitivity to change and the discriminant capacity, while preserving the historical clinometric properties of the unmodified POMA-G. Additionally, the mPOMA-G required no special instrumentation or camera angles, showed robust inter-rater reliability, and was well-correlated with other measures of clinically meaningful functionality.

TABLE 3
Modified Performance Oriented Mobility
Assessment - Gait (mPOMA-G) Assessment

| Assessment | | Observation | Score |
|---|---|---|---|
| 1 | Step length and height | | |
| | a) Right swing foot | Does not pass the left stance foot with step | =0 |
| | | Right heel passes the left stance foot | =1 |
| | | Right foot passes the left stance foot by at least the | =2 |
| | | length of individual's foot between the stance toe | |
| | | and swing heel | |
| | b) Right foot clear | Right foot does not clear floor completely with step | =0 |
| | | or raises foot by more than 1-2 inches | |
| | | Right foot completely clears floor | =1 |
| | c) Left swing foot | Does not pass the right stance foot with step | =0 |
| | | Left heel passes the right stance foot | =1 |
| | | Left foot passes the right stance foot by at least the | =2 |
| | | length of individual's foot between the stance toe | |
| | | and swing heel | |
| | d) Left foot clear | Left foot does not clear floor completely with step | =0 |
| | | or raises foot by more than 1-2 inches | |
| | | Left foot completely clears floor | =1 |
| 2 | Step symmetry | Right and left step length not equal (estimate) | =0 |
| | | Right and left step appear equal | =1 |
| 3 | Step continuity | Stopping or discontinuity between steps | =0 |
| | | Steps appear continuous unilaterally (observe | =1 |
| | | raising heel of 1 foot as heel of other foot touches | |
| | | the floor, unilaterally) or flat foot contact on stance | |
| | | limb when heel of other foot touches the floor | |
| | | bilaterally, no breaks or stops in stride | |
| | | Steps appear continuous bilaterally (observe raising | =2 |
| | | heel of 1 foot as heel of other foot touches the | |
| | | floor, bilaterally), no breaks or stops in stride, step | |
| | | lengths equal | |
| 4 | Trunk stability | Marked sway or uses walking aid. Marked sway = | =0 |
| | | moderate lateral flexion as the result of instability | |
| | | bilateral or unilateral | |
| | | No marked sway but compensatory patterns such | =1 |
| | | as trunk flexion, knee flexion, arm abduction or | |
| | | retraction to increase postural stability while | |
| | | walking | |
| | | No sway, no flexion, no use of arms, and no | =2 |
| | | walking aid | |
| 5 | Walk stance | Heels always apart, wide base of support utilized to | =0 |
| | | increase postural stability | |
| | | Heels intermittently apart | =1 |

| | |
|---|---|
| Gait score | /12 |

### Example 2

### HPP Patients Treated with Asfotase Alfa and Historical HPP Controls

All HPP patients included in the mPOMA-G analyses were required to have: 1) documented diagnosis of HPP with onset of symptoms months of age (i.e., childhood HPP), and 2) ≥2 videos of sufficient quality to assess gait between the ages of≥5 and 15 years. The either asfotase-alfa treated patients were enrolled in an ongoing, phase 2, open-label study, in which asfotase alfa was administered at a dosage of 6 mg/kg/week to these patients. Of the treated HPP patients, five HPP patients had data from videos both prior to and after initiation of treatment with asfotase alfa, and thus, a pre-treatment change in gait was compared to the on-treatment change in gait. The six HC HPP patients were enrolled in a non-interventional functional natural history study and served as the non-concurrent control group for the eight juvenile-onset patients treated with asfotase alfa for these gait analyses. All historical HPP control patients either declined participation in the treatment trials, or no longer met the age criteria.

Demographic and baseline characteristics for the patient groups are shown in Table 4. Across the groups, the majority of patients were white males. Overall, the HC patients and the all treated patients were similar with respect to age at HPP onset, baseline, and last gait assessment. HPP patients (n=5) with available pre-treatment data tended to be slightly younger at the onset of HPP symptoms and in their initial assessment compared to the HC group.

TABLE 4
Baseline characteristics for historical HPP control patients, patients with pre-treatment
and on-treatment gait data, and all treated patients (treatment with asfotase alfa).

| | Juvenile-Onset | | | | Juvenile-Onset | |
|---|---|---|---|---|---|---|
| | Historical HPP | | Juvenile-Onset | | All Treated | |
| | control | | Pre-& On-Treatment | | with Asfotase | |
| Variable | (N = 6) | | (N = 5) | | Alfa (N = 8) | |
| Age at Enrollment (months), n | ND ^{a} | | ND ^{b} | | 8 | |
| Mean (SD) | NA | | NA | | 101.70 | (23.936) |
| Median | NA | | NA | | 100.35 | |
| Min, Max | NA | | NA | | 72.3, 144.5 | |
| Age at Onset of HPP (months) n | 6 | | 5 | | 8 | |
| Mean (SD) | 16.5 | (11.24) | 14.59 | (4.319) | 15.25 | (4.027) |
| Median | 11.5 | | 12.00 | | 13.50 | |
| Min, Max | 8, 36 | | 12.0, 22.0 | | 12.0, 22.0 | |
| Age at Baseline Gait Assessment (years), n | 6 | | 5 | | 8 | |
| Mean (SD) | 7.10 | (2.208) | 6.68 | (1.839) | 8.58 | (1.972) |
| Median | 6.20 | | 6.30 | | 8.45 | |
| Min, Max | 5.3, 10.7 | | 4.3, 9.1 | | 6.2, 12.1 | |
| Age at Last Gait Assessment (years) | 6 | | 5 | | 8 | |
| Mean (SD) | 10.95 | (2.279) | 8.86 | (2.033) | 10.31 | (1.840) |
| Median | 11.05 | | 8.70 | | 10.20 | |
| Min, Max | 8.2, 14.9 | | 6.5, 12.1 | | 8.2, 14.1 | |
| Sex | | | | | | |
| Male, n (%) | 6 | (100.0) | 4 | (80.0) | 6 | (75.0) |
| Female, n (%) | 0 | (0.0) | 1 | (20.0) | 2 | (25.0) |
| Ethnicity | | | | | | |
| Hispanic or Latino, n (%) | 2 | (33.3) | 0 | | 1 | (12.5) |
| Not Hispanic or Latino, n (%) | 4 | (66.7) | 5 | (100.0) | 7 | (87.5) |
| Race | | | | | | |
| American Indian or Alaskan Native, n (%) | 0 | | 0 | | 0 | |
| Asian, n (%) | 0 | | 0 | | 0 | |
| Black or African American, n (%) | 0 | | 0 | | 0 | |
| Native Hawaiian or | 0 | | 0 | | 0 | |
| Other Pacific Islander, n (%) | | | | | | |
| White, n (%) | 6 | (100.0) | 5 | (100.0) | 8 | (100.0) |

| | | | |
|---|---|---|---|
| Other, n (%) | 0 | 0 | 0 |

Abbreviations:
max = maximum,
min = minimum,
ND = not done,
SD = standard deviation
^{a} Age at enrollment was not applicable for the historical HPP control patients because data were abstracted retrospectively from medical records.
^{b} Age at enrollment was not calculated for the sub-set of treated patients with pre- and post-treatment data.

The medical history most relevant to gait for the treated and HC patients is shown in Table 5. All eight HPP patients treated with asfotase alfa and all six HC patients had a history of gait disturbance. Bone deformity (bowing of the long bones), bone pain, and muscular weakness were the most frequent symptoms reported across the patient groups. Gait deficits were also noted at the baseline assessment of the mPOMA-G. The most common deficits (foot clearance, stance, proximal sway/trunk, and step length) implicate loss of proximal muscle strength in the pathophysiology of HPP and the subsequent development of compensatory strategies in HPP patients (e.g., waddling gait pattern and increased base of support).

TABLE 5
Relevant HPP-specific medical history for patient populations.

| | Juvenile-Onset | | Juvenile-Onset | | Juvenile-Onset | |
|---|---|---|---|---|---|---|
| | Historical HPP | | Pre-& On-Treatment | | All Treated | |
| | (N = 6) | | (N = 5) | | (N = 8) | |
| | n (%) | | n (%) | | n (%) | |
| Arthralgia/Joint Pain | 3 | (50.0) | 1 | (20.0) | 2 | (25.0) |
| Joint pain that limited daily activities | 3 | (50.0) | — | | — | |
| Joint pain that required pain medications for | 3 | (50.0) | — | | — | |
| management | | | | | | |
| Bone deformity (Bowing of the long bones) | 6 | (100.0) | 2 | (40.0) | 3 | (37.5) |
| Bone pain | 3 | (50.0) | 2 | (40.0) | 4 | (50.0) |
| Bone pain that limited daily activities | 3 | (50.0) | 1 | (20.0) | 2 | (25.0) |
| Bone pain that required pain medications for | 2 | (33.3) | 1 | (20.0) | 2 | (25.0) |
| management | | | | | | |
| Fracture | 3 | (50.0) | 1 | (20.0) | 2 | (25.0) |
| Gait disturbance | 6 | (100.0) | 5 | (100.0) | 8 | (100.0) |
| Unusual gait | 5 | (83.3) | — | | — | |
| Waddling gait | 4 | (66.7) | — | | — | |
| Muscular weakness | 3 | (50.0) | 3 | (60.0) | 5 | (62.5) |
| Muscle weakness that limited daily | 3 | (50.0) | — | | — | |
| activities | | | | | | |
| Myalgia/Muscle Pain | 2 | (33.3) | 3 | (60.0) | 4 | (50.0) |
| Muscle pain that limited daily activities | 2 | (33.3) | — | | — | |
| Muscle pain that required pain medications | 1 | (16.7) | — | | — | |
| for management | | | | | | |
| Scoliosis | 1 | (16.7) | — | | — | |
| Gait Deficits (mPOMA-G) ^{a} | 6 | (100.0) ^{a} | 5 | (100.0) ^{b} | 8 | (100.0) ^{a} |
| Step length (swing) | 4 | (66.7) | 4 | (80.0) | 7 | (87.5) |
| Steppage (foot clearance) | 5 | (83.3) | 4 | (80.0) | 8 | (100.0) |
| Proximal sway/trunk | 6 | (100.0) | 5 | (100.0) | 8 | (100.0) |
| Stance | 2 | (33.3) | 2 | (40.0) | 6 | (75.0) |

^{a} Gait deficit data based on mPOMA-G scores at baseline.
^{b} Gait deficit data based on mPOMA-G scores at historical time point.

### Example 3

### Collection of Gait Data to Determine mPOMA-G of Patient Populations

Three qualified and trained physical therapists scored videos of walking using the mPOMA-G. The raters did not participate in caring for enrolled patients and were masked to patient identifiers and the dates of video recording. All raters were provided an Independent Rater Manual describing the process for scoring the functional videos in support of their training. Visible faces on the videos were permanently blurred, and videos were masked and randomized prior to scoring

The mPOMA-G score for each available time point was obtained by summing the median score (across the 3 raters) for each individual component at that time point. For the HC patients, baseline was defined as the earliest available mPOMA-G score within the period from 5 to 15 years of age (allowing for a 2-week window around these ages). Change from baseline was computed for each post-baseline time point. The last available assessment was used for the comparison.

For asfotase alfa-treated patients, the last available assessment was defined as the last time point for which the time from baseline was less than or equal to the median time from baseline among mPOMA-G analysis results from HC patients. The time point for the last assessment in treated patients was always less than the median time from baseline in the HC patients; therefore, no data were censored.

Rate of change in mPOMA-G score was compared between treated and HC patients (primary endpoint) using a Wilcoxon rank-sum test with a 2-sided alpha of 0.05, an exact method for determining the P-value. For each patient, the rate of change was computed by dividing the change from baseline to last assessment available in mPOMA-G score by the time (in years) from baseline to last assessment. For asfotase alfa-treated HPP patients with a video available prior to the baseline assessment, rate of change per year in mPOMA-G score was determined for the time period between pre-baseline and the baseline assessment in addition to between baseline and the last assessment on treatment. Within-group changes from baseline and from pre-treatment assessments were evaluated using the Wilcoxon signed-rank statistical test.

### Example 4

### Validation of mPOMA-G to Assess Gait Improvements in HPP Patients

Intraclass correlation coefficients (ICCs) were calculated to assess overall inter-rater and intra-rater agreement. Raters scored videos of children with childhood and adolescent HPP (≥6 months of age) from 2 clinical studies (asfotase alfa clinical trial participants, n=8; natural history study participants, n=6), as is described in Example 2. For inter-rater ICC, a two-way random effects analysis of variance model with terms for subject-visit, rater, and the interaction between subject-visit and rater was used. For intra-rater ICC, a two-way random effects analysis of variance model with terms for subject-visit-rater, review (original or retest), and the interaction between subject visit-rater and review was used. The null hypothesis was tested with a 0.025 level of significance, and was rejected only if ICC>0. Inter-rater agreement across all visits was excellent (ICC=0.76, p<0.0001, number of observations=192). Likewise, intra-rater agreement across all visits was also excellent (ICC=0.76, p<0.001, number of observations=192).

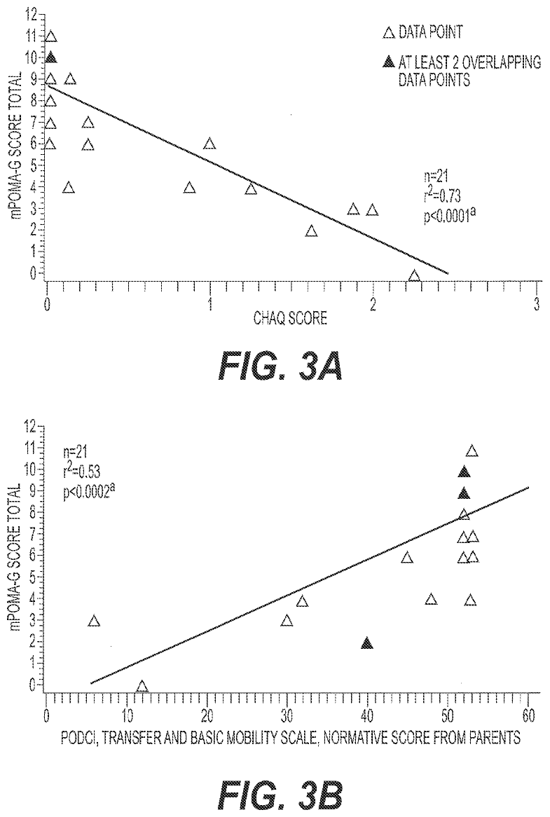

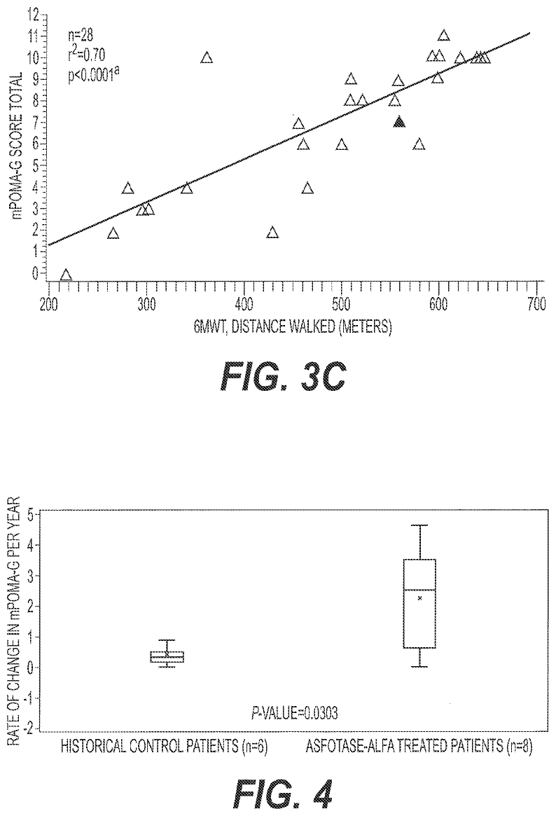

For concurrent validity analysis, linear regression was performed to determine the relationship between mPOMA-G scores and other clinical outcome measures in children and adolescents treated with asfotase alfa (n=5), which included the Childhood Health Assessment Questionnaire (CHAQ) Disability Index, Pediatric Outcomes Data Collection Instrument (PODCI) Transfer and Mobility Scale (normative score reported by parents), and the Six Minute Walk Test (6MWT; distance walked in meters). The 6MWT was chosen because this metric measure ambulatory capacity, and the PODCI and CHAQ were chosen because these metrics measure ability to participate in normal activities of daily living (e.g., climbing stairs and getting onto a bus) and community sports and recreation. There were strong correlations between the mPOMA-G scores of HPP patients and HC patients with measures of ability to perform activities of daily living as assessed using the CHAQ (FIG. 3A) and PODCI (FIG. 3B) in addition to measures of physical function as assessed using the 6MWT (FIG. 3C).

### Example 5

### mPOMA-G Minimum Clinically Important Difference

A minimum clinically meaningful difference (MCID) in mPOMA-G score was prospectively determined based on the relationship between mPOMA-G score and other endpoints for which reports of clinically meaningful changes were available, i.e., the CHAQ disability index (change of −0.13) (Dempster, H., et al., Arthritis & Rheumatism 44(8):1768-74 (2001)), PODCI transfer and basic mobility scale normative score from parents (change of 4.5) (see Henricson, E., et al., PLOS Currents Muscular Dystrophy 2013, 1-21, 6MWT and Duchenne muscular dystrophy; Oeffinger, D., et al., Dev Med & Child Neurol 50: 918-925 (2008), ambulatory cerebral palsy in children), and 6MWT distance walked (30 meters) (McDonald, C.M. et al., Muscle Nerve 48:357-68 (2013)). Using data from the 5 infantile-onset HPP patients (prior to analyzing the juvenile-onset data), separate linear regressions were performed for the mPOMA-G with the CHAQ (r²=0.73 [21 data points]), PODCI (r²=0.53 [21 data points]), and 6MWT (r²=0.70 [28 data points]).

Using the resulting linear equations, a clinically meaningful change of −0.13 on the CHAQ corresponded with a change of 0.45 in mPOMA-G score, a clinically meaningful change of 4.5 on the PODCI Test of Motor Proficiency corresponded with a change of 0.76 in mPOMA-G score, and a clinically meaningful change of 30 meters on the 6MWT correlated with a change of 0.59 in mPOMA-G score. Of these, the CHAQ and PODCI were used because they are disability measures for which minimally clinically important differences in pediatric populations were expected to be relatively disease-independent, and therefore better for correlative analysis. The correlations with minimal clinically important 6MWT changes were also included for completeness. Based on results of all three analyses, a change of 0.6 in mPOMA-G score was prospectively identified as a clinically meaningful change.

These data demonstrate that mPOMA-G analysis is a reliable method for detecting clinically significant impairments in gait, as well as clinically significant changes in impairments in gait, using videos of children and adolescents with HPP. Accordingly, mPOMA-G score showed strong concurrent association with physical function and patient-reported disability when performing activities of daily living.

### Example 6

### Analysis of Physical Function and Gait Impairments in Historical HPP Controls

The HC patients varied in mobility from the most severe patient, who required a walker or wheelchair up to 50% of the time, to the highest functioning patient, who could participate in community sports. For all patients, activity was limited by fatigue and/or pain. Some patients had challenges in keeping up with healthy peers. Muscle weakness was noted using manual muscle testing in these patients and was most commonly seen in hip extensors and hip abductors.

Three raters assessed videos of the HC patients for gait descriptors including genu varum/valgus, position of knees and hips at terminal stance, and use of orthotics; running descriptors including arm swing, presence of Trendelenburg, steppage, circumduction or scissoring, foot contact and push off pattern, and presence/absence of period of flight; the ability to stand on one foot; and the ability to rise from floor to standing without use of hands.

Genu valgum or “knock-knee” was observed in 67% (4/6) HC patients at first assessment, and all six HC patients exhibited genu valgum at last assessment. A running pattern that utilized strategies to compensate for decreased proximal stability and weakness was observed in all six HC patients. At last assessment, 67% of HC patients were unable to achieve a period of flight, indicating that actual running was not achieved. According to the Peabody Developmental Motor Scales (PDMS-2), the mean normative age to acquire running with a period of flight with both feet in the air is approximately 1.6 years, while a mature running pattern is typically acquired by approximately 3.4 years of age. For a description of the PDMS-2 scales, see van Hartingsveldt et al. (Occup. Ther. Int 12(1): 1-13, 2005), hereby incorporated by reference in its entirety.

Time standing on one foot was lower in all HCs than has been reported previously in healthy age-matched peers, which was 1.5 to 5.6 sec in all six (100%) HCs, which were older than 5.3 years at first assessment. For reference, the PDMS-2 mean normative age to achieve standing on one foot for 5 seconds is approximately 3.8 years. Trendelenburg signs (indicative of weak abductor muscles of the hip) were noted in all six HCs. All HCs required self-support with one or two hands to transition from the floor to standing, further indicating proximal instability or weakness.

Gait of the HCs was then assessed using the mPOMA-G, as described in Examples 1-3. Each HC had one pair of videos for gait evaluation. The median (min, max) time between first assessment and last assessment was 49.2 (24, 71) months. All HCs demonstrated measurable gait impairments at first assessment, with mPOMA-G scores ranging from 3 to 11 (median 6; Table 6). All six HCs retained gait impairments from first assessment to last assessment, with mPOMA-G scores ranging from 4 to 11 (median 7.5) at last assessment. There was no consistent pattern of change in mPOMA-G components (Table 7).

TABLE 6
Gait assessment of HPP historical HPP controls.

| Median | | | |
|---|---|---|---|
| (min, max), | First | Last | Change from |
| n = 6 | Assessment | Assessment | First Assessment |
| Age at | 6.2 (5.3, 10.7) | 11.1 (8.2, 14.9) | 4.1 (2.1, 5.9) |
| assessment, | | | |
| years | | | |
| mPOMA-G | 6.00 (3.0, 11.0) | 7.50 (4.0, 11.01) | 1.50 (0.0, 2.0), |
| score | | | P = 0.0625 |

P-value based on nonparametric sign test to determine whether the median change from baseline differed from 0.

TABLE 7
Change in mPOMA-G components* of historical HPP controls.

| | | | | Number of |
|---|---|---|---|---|
| | Median score (min, max) | | | patients |
| Assessment (min, max | First | Last | | with change |
| possible points) | Assessment | Assessment | | (n = 6) |
| Right foot clear (0, 1) | 0 (0, 1) | 0 | (0, 1) | 0 |
| Left foot clear (0, 1) | 0 (0, 1) | 0 | (0, 1) | 0 |
| Right swing foot (0, 2) | 1 (1, 2) | 1.5 | (1, 2) | 1 |
| Left swing foot (0, 2) | 1 (1, 2) | 1.5 | (1, 2) | 1 |
| Step symmetry (0, 1) | 1 (0, 1) | 1 | (0, 1) | 0 |
| Step continuity (0, 2) | 1 (1, 2) | 1.5 | (1, 2) | 1 |
| Trunk (0, 2) | 1 (0, 1) | 1 | (0, 2) | 2 |
| Walk stance (0, 1) | 1 (0, 1) | 1 | (0, 1) | 2 |

*Component score for each patient is determined as the median score of 3 raters.

Step length and continuity, steppage gait pattern, and marked trunk sway were among commonly observed gait deviations for the HCs, which persisted from first assessment to last assessment (Table 8).

TABLE 8
Individual components of mPOMA-G scores observed by raters
at First and Last Assessments of historical HPP controls.

| Patient | First Assessment | Last Assessment |
|---|---|---|
| 1 | Gait deviations in step length and continuity, and a | Gait deviations in step length and |
| | steppage gait pattern | continuity, and a steppage gait pattern |
| | Widened stance and marked trunk sway | Narrower stance and mild trunk sway |
| 2 | Gait deviations in step length and continuity | Gait deviations in step length and continuity |
| | Steppage gait pattern | Steppage gait pattern |
| | Marked trunk sway | Marked trunk sway |
| | Widening of the base of support to increase stability | Widening of the base of support to increase |
| | Step length asymmetry (2/3 raters) | stability |
| | | No step length asymmetry (3 raters) |
| 3 | Mild trunk sway or presence of compensatory arm | Mild trunk sway or presence of |
| | patterns to compensate for proximal instability | compensatory arm patterns to compensate |
| | | for proximal instability |
| 4 | Steppage gait pattern | Steppage gait pattern |
| | Mild trunk sway | Mild trunk sway |
| | Stepping just past the stationary foot bilaterally | Stepping past the stance foot by at least |
| | | 1 foot length bilaterally |
| 5 | Gait deviations in step length and continuity | Gait deviations in step length and continuity |
| | Steppage gait pattern | Steppage gait pattern |
| | Marked trunk sway | Marked trunk sway |
| | Widening of the base of support | Heels intermittently widened or almost |
| | | touching while walking |
| 6 | Steppage gait pattern | Steppage gait pattern (2/3 raters) |
| | Lack of step continuity (1/3 raters) | Normal step continuity (3 raters) |
| | Mild trunk sway (1/3 raters) | Absence of trunk sway (3 raters) |

Bold text, an improvement has been observed from First to Last Assessment.

These results indicate that proximal muscle weakness was prevalent and persistent in the HCs, as indicated by gait descriptors, functional mobility items, and mPOMA-G scores. All HCs had clinically significant gait impairment that persisted throughout the study.

### Example 7

### Comparative Gait Analyses using mPOMA-G of HPP Patients and Historical HPP Controls

Children and adolescent HPP patients treated with asfotase alfa demonstrated a statistically significant improvement in rate of change per year in mPOMA-G score from baseline to last assessment compared with HCs (FIG. 4 and Table 9). The median (range) rate of change per year in asfotase alfa-treated patients was 2.51 (0.0, 4.6) compared with 0.33 (0.0, 0.9) in HCs (p=0.0303, Wilcoxon rank sum test (exact method)). Rate of change using the POMA-G score was directionally the same as for the mPOMA-G score, with treated patients demonstrating a greater rate of change compared to the HCs.

TABLE 9
Rate of change per year from baseline to last assessment
of mPOMA-G score in asfotase alfa-treated versus historical
HPP control patients with juvenile-onset HPP.

| | | |
|---|---|---|
| | Juvenile-Onset | |
| | Historical | Juvenile-Onset |
| | HPP control | Treated |
| Statistic | (N = 6) | (N = 8) |

Baseline mPOMA-G

| N | | | 8 | |
|---|---|---|---|---|
| Mean (SD) | 6.3 | (2.94) | 4.6 | (2.33) |
| Median (min, max) | 6.0 | (3, 11) | 4.0 | (2, 9) |

mPOMA-G Rate of Change (per year)

| N | 6 | | 8 | |
|---|---|---|---|---|
| Mean (SD) | 0.37 | (0.306) | 2.25 | (1.672) |
| Median (min, max) | 0.33 | (0.0, 0.9) | 2.51 | (0.0, 4.6) |

| | | |
|---|---|---|
| p-value (within group) ^{a} | 0.0625 | 0.0156 |

| | |
|---|---|
| p-value (between group) ^{b} | 0.0303 |

Abbreviations:
max = maximum,
min = minimum,
SD = standard deviation
^{a} Within group comparison of change from Baseline to Last Assessment based on Wilcoxon signed rank test.
^{b} Between group comparison of change from Baseline to Last Assessment based on Wilcoxon rank sum test (exact method).

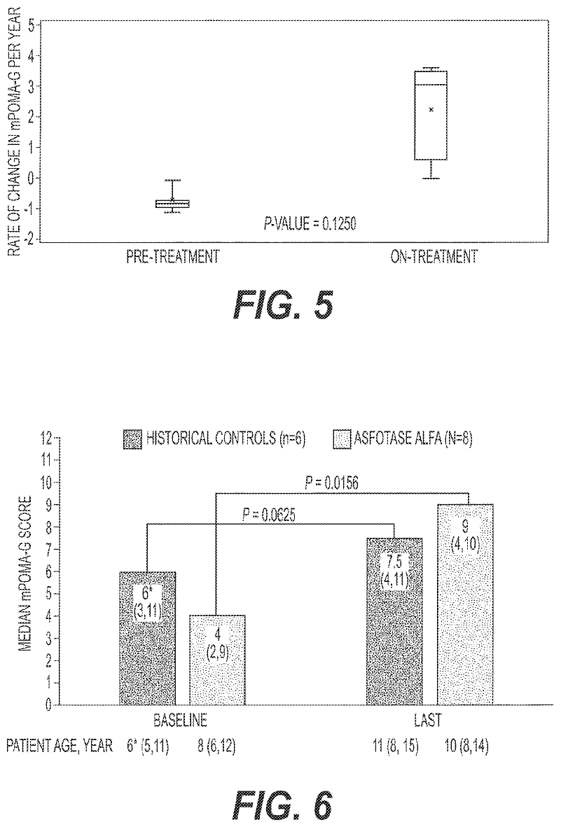

For HPP patients treated with asfotase alfa that had a pre-treatment video available, rate of change per year in mPOMA-G score was determined for the time period prior to and after receiving treatment with asfotase alfa. Deterioration in gait was observed prior to starting treatment with asfotase alfa (median rate of change/year of −0.72); whereas, the median on-treatment rate of change per year was 3.01 (0.0, 3.6) (FIG. 5 and Table 10). These results were consistent with the results of the comparison with HCs, with the treated group showing improvements in gait when compared with either historical or pre-treatment controls.

TABLE 10
Rate of change per year in mPOMA-G score pre-treatment versus
on-treatment in juvenile-onset HPP patients (N = 5).

| | | |
|---|---|---|
| | Pre-Treatment | On-Treatment |
| Statistic | (N = 5) | (N = 5) |

mPOMA-G Rate of Change (per year)

| N | 5 | | 5 | |
|---|---|---|---|---|
| Mean (SD) | −0.67 | (0.401) | 2.14 | (1.669) |
| Median (min, max) | −0.72 | (−1.0, 0.0) | 3.01 | (0.0, 3.6) |

| | | |
|---|---|---|
| p-value | 0.1250 | 0.1250 |

Abbreviations:
max = maximum,
min = minimum,
SD = standard deviation
Within group comparison of change from Baseline to Last Assessment based on Wilcoxon signed rank test.

Patients treated with asfotase alfa had an absolute median per patient change from baseline in mPOMA-G score that was twice as great as HCs (median change of 3.0 versus 1.5, respectively; FIG. 6 and Table 11). While both groups exceeded the pre-identified mPOMA-G minimally clinical important difference (MCID) of 0.6, patients treated with asfotase alfa had a median improvement in score twice as great as the HCs.

TABLE 11
Change from baseline to last assessment of mPOMA-G score
for asfotase alfa-treated versus historical HPP controls

| | Juvenile-Onset | | | | Juvenile-Onset | | | |
|---|---|---|---|---|---|---|---|---|
| | Historical HPP | | | | Treated | | | |
| | control (N = 6) | | | | (N = 8) | | | |
| | Actual | | Change | | Actual | | Change | |
| Statistic | Score | | from BL | | Score | | from BL | |
| Baseline | | | | | | | | |
| N | 6 | | — | | 8 | | — | |
| Mean (SD) | 6.3 | (2.94) | — | | 4.6 | (2.33) | — | |
| Median | 6.0 | (3, 11) | — | | 4.0 | (2, 9) | — | |
| (min, max) | | | | | | | | |
| Last | | | | | | | | |
| Assessment | | | | | | | | |
| N | 6 | | 6 | | 8 | | 8 | |
| Mean (SD) | 7.7 | (2.73) | 1.3 | (0.82) | 7.9 | (2.17) | 3.3 | (2.60) |
| Median | 7.5 | (4, 11) | 1.5 | (0, 2) | 9.0 | (4, 10) | 3.0 | (0, 7) |
| (min, max) | | | | | | | | |

| | | |
|---|---|---|
| p-value | 0.0625 | 0.0156 |
| (within | | |
| group)^{a} | | |

| | |
|---|---|
| p-value | 0.2561 |
| (between | |
| group) ^{b} | |

^{a}Within group comparison of change from Baseline to Last Assessment based on Wilcoxon signed rank test.
^{b} Between group comparison of change from Baseline to Last Assessment based on Wilcoxon rank sum test (exact method)
Abbreviations:
BL = Baseline:
max = maximum,
min = minimum,
SD = standard deviation

The most common deficits in gait observed at baseline for treated patients and HCs (foot clearance, stance, proximal sway/trunk, and step length) implicate lack of proximal muscle strength as a complication of HPP, which results in compensatory gait abnormalities (e.g., waddling gait pattern and increased base of support). Following asfotase alfa treatment, components of the gait analysis showing the greatest incidence of improvement were step length and stance (Table 12).

TABLE 12
Incidence of the separate components of mPOMA-G prior to and
following treatment of HPP patients with asfotase alfa.

| | Baseline | | |
|---|---|---|---|
| | Historical | Treated Patients | |
| | Controls | (n = 8) n (%) | |
| Gait Deficits | (n = 6) | | Post-Asfotase |
| (mPOMA-G) | n (%) | Baseline | Alfa |
| Reduced step length (swing) | 4 (66.7) | 7 (87.5) | 1 (12.5) |
| Steppage (foot clearance) | 5 (83.3) | 8 (100.0) | 7 (87.5) |
| Widened stance | 2 (33.3) | 6 (75.0) | 2 (25) |

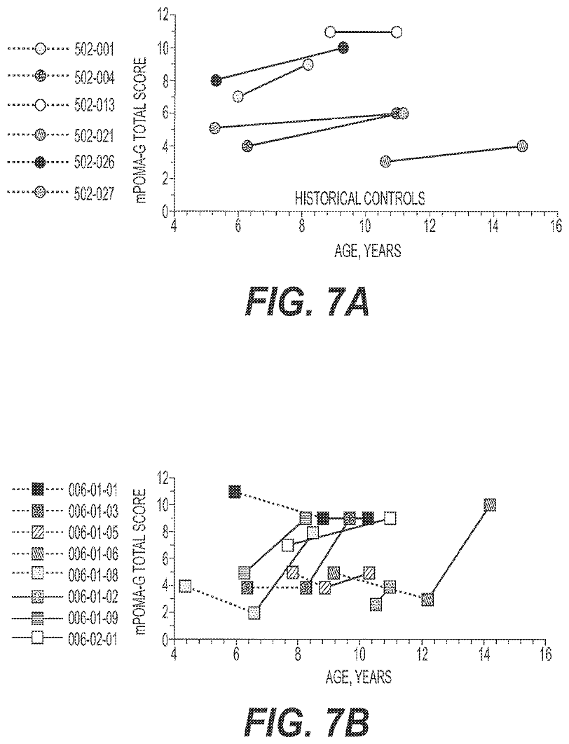

Baseline to last assessment for each patient at the two time points was also compared relative to patient age (FIGS. 7A-7B). The range of ages was similar for both the asfotase alfa-treated and historical HPP control patients with the minimum and maximum ages at baseline ranging from 6 to 12 years in treated patients compared with 5 to 11 years in HCs and at last assessment ranging from 8 to 14 years for asfotase alfa-treated patients compared to 8 to 15 years for historical HPP control patients. Neither age at baseline nor age at last assessment was significantly different between the two groups.

Importantly, comparison of individual changes in mPOMA-G from baseline supports the conclusion that treatment with asfotase alfa improves performance-oriented mobility in child and adolescent patients with juvenile-onset HPP. As shown in FIGS. 7A-7B, one patient in the historical HPP control group and one patient in the asfotase-alfa treated group maintained the same score at baseline and last assessment. Changes from baseline in the remaining five historical HPP control patients were relatively small, with two patients increasing by 1 point, 3 patients increasing by 2 points, and no patients experiencing improvements greater than 2 points, for a median change from baseline of 1.5 points. In contrast, for the remaining seven (88%) asfotase alfa-treated patients, two patients improved from baseline to last assessment by 1 point, two patients improved by 4 points, one patient improved by 5 points, and two patients improved by 6 points, for a median change from baseline of 3.0 points. POMA-G results were consistent in trend but were of less magnitude than those from the HPP-specific mPOMA-G.

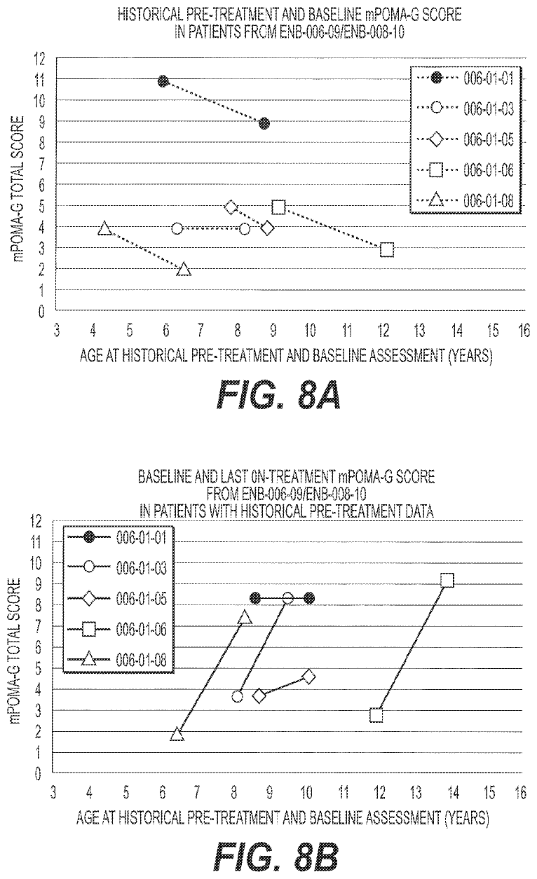

In contrast to the historical HPP control data, where five out of six HCs demonstrated some improvement in gait over time, four out of five asfotase alfa-treated patients with pre-treatment gait data demonstrated increased disability prior to treatment (FIG. 8A). Prior to administration of asfotase alfa, the median change in mPOMA-G score was −2.0 (range: −2, 0); whereas, after starting treatment the median improvement was 5.0 (range: 0, 7) at the last assessment (FIG. 8B and Table 13). Both the median pre-treatment deterioration and the on-treatment improvement in gait markedly exceeded the pre-identified MCID of 0.6.

TABLE 13
Change in mPOMA-G score pre-treatment versus on-
treatment in asfotase alfa-treated patients.

| | Pre-Treatment (N = 5) | | On-Treatment (N = 5) | |
|---|---|---|---|---|
| | Actual | Change | Actual | Change |
| | Score | from BL | Score | from BL |
| | Earliest Historical | | Treatment | |
| Statistic | Result | | Baseline | |
| n | 5 | — | 5 | — |
| Mean (SD) | 5.8 (2.95) | — | 4.4 (2.70) | — |
| Median (min, max) | 5.0 (4, 11) | — | 4.0 (2, 9) | — |
| | Treatment | | Treatment Last | |
| | Baseline | | Assessment | |
| n | 5 | 5 | 5 | 5 |
| Mean (SD) | 4.4 (2.70) | −1.4 (0.89) | 8.2 (1.92) | 3.8 (3.11) |
| Median (min, max) | 4.0 (2, 9) | −2.0 (−2, 0) | 9.0 (5, 10) | 5.0 (0, 7) |

| | | |
|---|---|---|
| p-value | 0.1250 | 0.1250 |
| (within | | |
| group) ^{a} | | |

^{a} Within group comparison of change from Baseline to Last Assessment based on Wilcoxon signed rank test.
Abbreviations:
BL = Baseline;
max = maximum,
min = minimum,
SD = standard deviation

Regardless of age at the earliest historical assessment, four (80.0%) asfotase-alfa treated patients exhibited increased gait disability prior to treatment and one patient remained stable. Additionally, regardless of age at treatment-onset, four (80.0%) asfotase-alfa treated patients demonstrated improved gait after treatment with asfotase alfa and one patient remained stable. In addition, the improvement observed in gait score exceeded the MCID for all four asfotase-alfa treated patients.

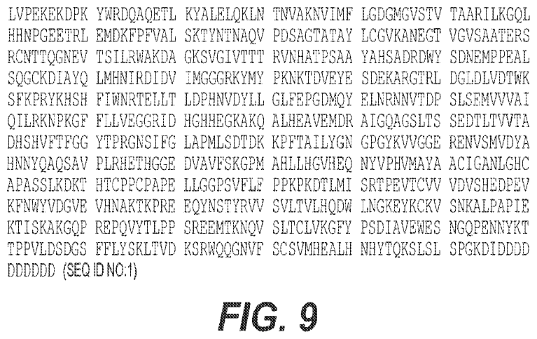

These data show that the mPOMA-G analysis and score accurately quantify both deficits and changes in gait in children and adolescents with HPP. Moreover, children and adolescents with impaired gait due to HPP demonstrated clinically significant improvement in functional mobility when treated with asfotase alfa compared to these HPP patients prior to treatment with asfotase alfa and HCs. Thus, asfotase alfa (SEQ ID NO: 1; FIG. 9) can be used effectively to treat HPP, its symptoms, and decreased physical function associated therewith in patients of about 5 to about 15 years of age for an extended treatment period, such as at least one year, at least two years, at least three years, at least four years, at least five years, at least six years, at least seven years, at least eight years, at least nine years, at least ten years, or longer than ten years, such as for the lifetime of the patient.

### Example 8

### Optimization of Asfotase Alfa Dosage to Treat Gait Impairments in HPP Patients

The dosage of asfotase alfa (SEQ ID NO: 1) can be increased to, e.g., 9 mg/kg/wk, when a child or adolescent with HPP has an average mPOMA-G score of less than about 12 after administration of asfotase alfa at a dosage of 6 mg/kg/wk for a treatment period of at least one year. For instance, the average mPOMA-G score of the child or adolescent will increase to about 8 to about 11 (e.g., 8, 9, 10, or 11) after being treated with an sALP at a dosage of 6 mg/kg/wk for a treatment period of at least one year. The sALP can then be administered at a dosage of, e.g., 9 mg/kg/wk until the child or adolescent exhibits further improvements in gait, as determining using the mPOMA-G analysis and score. In particular, the average mPOMA-G score of the child or adolescent can increase from about 8 to about 12 after administration of an sALP at a dosage of 9 mg/kg/wk for a treatment period of at least one year, such as at least two years, at least three years, at least four years, at least five years, at least six years, at least seven years, at least eight years, at least nine years, at least ten years, or longer than ten years (e.g., the lifetime of the patient).

### OTHER EMBODIMENTS

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the claimed invention. Although the disclosure has been described in connection with specific embodiments, it will be understood that it is capable of further modifications and that the invention as claimed should not be unduly limited to such specific embodiments.

1. A method of treating hypophosphatasia (HPP) in a subject of about 5 to about 15 years of age having an average modified Performance-Oriented Mobility Assessment—Gait (mPOMA-G) score of about 5 or less, wherein said method comprises administering a soluble alkaline phosphatase (sALP) to the subject at a dosage providing about 6 mg/kg/week of the sALP, wherein the sALP comprises an amino acid sequence having at least 95% sequence identity to the amino acid sequence of SEQ ID NO: 1, and wherein administration of the sALP for a treatment period of at least one year results in an increase in the average mPOMA-G score of at least about 0.6, particularly at least about 1.0 or more.

2. The method of claim 1, wherein the average mPOMA-G score of the subject is determined relative to an average mPOMA-G score of an untreated subject of about 5 to about 15 years of age having HPP or the average mPOMA-G score of the subject is determined relative to an average mPOMA-G score of a healthy subject of about 5 to about 15 years of age.

3. The method of claim 1, wherein the method further comprises performing an mPOMA-G analysis.

4. The method of claim 3, wherein at least one of:
(a) the mPOMA-G analysis is performed daily, one or more times per week, weekly, one or more times per month, monthly, every six months, one or more times per year, yearly, every two years, or every three years; and
(b) the mPOMA-G analysis comprises one or more gait assessments selected from the group consisting of trunk sway, walking stance, step length and height, step symmetry, and step continuity.

5. The method of claim 4, wherein at least one of:
(a) trunk sway comprises measuring at least one of marked sway, use of walking aids, arm abduction, trunk flexion, and excessive knee flexion;
(b) walking stance comprises measuring distance of heels;
(c) step length and height comprise measuring right swing foot, right foot clear, left swing foot, and left foot clear;
(d) step symmetry comprises measuring right step length and left step length and comparing right step length to left step length; and
(e) step continuity comprises measuring stopping between steps or discontinuity between steps, wherein preferably discontinuity between steps is measured through evaluation of heel off in terminal stance on one foot at the same time as initial contact of heel strike on the opposite foot.

6. The method of claim 1, wherein:
(a) the sALP is administered for a treatment period of at least two years, at least three years, at least four years, at least five years, at least six years, at least seven years, at least eight years, at least nine years, at least ten years, or longer; and
(b) the average mPOMA-G score of the subject increases during the second treatment period to about 7.5, about 8, about 8.5, about 9, about 9.5, about 10, about 10.5, about 11, about 11.5, or about 12 after administration of the sALP.

7. The method of claim 6, wherein the average mPOMA-G score of the subject increases to about 9 after administration of the sALP.

8. The method of claim 1, wherein at least one of:
(a) prior to administration of the sALP, the subject exhibits one or more gait impairments selected from the group consisting of reduced step length, reduced step continuity, reduced foot clearance, foot clearance that exceeds about 1 or 2 inches off surface, and widened stance; and
(b) after administration of the sALP, the subject exhibits an improvement in one or more gait impairments selected from the group consisting of reduced step length, reduced step continuity, reduced foot clearance, foot clearance that exceeds about 1 or 2 inches off surface, and widened stance.

9. The method of claim 1, wherein at least one of:
(a) the increase in the average mPOMA-G score is sustained throughout a period during which the subject is treated with the sALP;
(b) a rate of change per year of the average mPOMA-G score is about 2.5;
(c) the subject has juvenile-onset HPP;
(d) the method further comprises performing at least one of a Six Minute Walk Test (6MWT), a Child Health Assessment Questionnaire (CHAO), and a Pediatric Outcomes Data Collection Instrument (PODCI);
(e) the subject exhibits an increase in activities of daily living (ADL) after administration of the sALP;
(f) the subject exhibits an improvement in walking ability;
(g) the subject has not been previously administered the sALP; and
(h) the subject is a child or an adolescent.

10. The method of claim 9, wherein the increase in ADL is determined from a CHAQ disability index score or PODCI transfer and mobility scale score of the subject or wherein the improvement in walking ability is determined from a 6MWT distance of the subject.

11. The method of claim 1, wherein at least one of:
(a) the sALP is formulated for daily or weekly administration;
(b) the sALP is administered twice a week, three times a week, four times a week, five times a week, six times a week, or seven times a week;
(c) the sALP is administered at a dosage of 2 mg/kg for administration three times a week, a dosage of 3 mg/kg for administration three times a week, or a dosage of 1 mg/kg for administration six times a week;
(d) the sALP is administered on consecutive or alternating days;
(e) the sALP is administered as a pharmaceutical composition with at least one pharmaceutically acceptable carrier; and
(f) the sALP is administered by at least one route selected from the group consisting of subcutaneous, intramuscular, intravenous, oral, nasal, sublingual, intrathecal, and intradermal; and
(g) the sALP comprises or consists of the amino acid sequence of SEQ ID NO: 1.

12. The method of claim 11, wherein the at least one pharmaceutically acceptable carrier is saline or comprises sodium chloride and sodium phosphate.

13. The method of claim 12, wherein the at least one pharmaceutically acceptable carrier comprises 150 mM sodium chloride and 25 mM sodium phosphate.

14. The method of claim 1, wherein the subject exhibits one or more symptoms of HPP selected from the group consisting of gait disturbance, bone deformity, joint pain, bone pain, bone fracture, muscle weakness, muscle pain, rickets, premature loss of deciduous teeth, incomplete bone mineralization, elevated blood and/or urine levels of phosphoethanolamine (PEA), elevated blood and/or urine levels of inorganic pyrophosphate (PPi), elevated blood and/or urine levels of pyridoxal 5′-phosphate (PLP), hypomineralization, rachitic ribs, hypercalciuria, short stature, HPP-related seizure, inadequate weight gain, craniosynostosis, and calcium pyrophosphate dihydrate crystal deposition.

15. The method of claim 14, wherein the subject exhibits an improvement in the one or more symptoms of HPP after administration of the sALP.

16. The method of claim 1, wherein at least one of:
(a) the sALP is physiologically active toward PEA, PPi, and PLP;
(b) the sALP is catalytically competent to improve skeletal mineralization in bone;
(c) the sALP is the soluble extracellular domain of an alkaline phosphatase;
(d) the method further comprises determining sALP activity in a serum and/or blood sample from the subject of about 5 to about 15 years of age;
(e) administration of the sALP for the second treatment period of at least one year results in an increase in the average mPOMA-G score of at least about 2.5 or more;
(f) the mPOMA-G analysis is performed before administration of the sALP; and
(g) the mPOMA-G analysis is performed after administration of the sALP.

17. The method of claim 16, wherein at least one of:
(a) the determination of sALP activity comprises measuring at least one of phosphoethanolamine (PEA), inorganic pyrophosphate (PPD, and pyridoxal 5′-phosphate (PLP) in at least one of serum and blood from the subject of about 5 to about 15 years of age; and
(b) the dose of the sALP is increased if the mPOMA-G score does not increase by at least about 0.6, 1.0, or 2.5 after the treatment period of at least one year.

18. The method of claim 17, wherein the dose is increased to about 9 mg/kg/week.

19. The method of claim 1, wherein the subject exhibits decreased reliance on an assistive device for mobility after administration of the sALP.

20. The method of claim 19, wherein the assistive device for mobility is selected from the group consisting of a wheelchair, braces, crutches, and orthotics.


SEQUENCE LISTING (20 sequences)

SEQ ID NO: 1 - protein, 726 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 2 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 3 - protein, 524 aa, Homo sapiens [sequence omitted]

SEQ ID NO: 4 - protein, 524 aa, Homo sapiens; identical to SEQ ID NO: 2

SEQ ID NO: 5 - protein, 524 aa, Homo sapiens; identical to SEQ ID NO: 2

SEQ ID NO: 6 - protein, 524 aa, Homo sapiens [sequence omitted]

SEQ ID NO: 7 - protein, 652 aa, Macaca mulatta
MPTVKTKQESHAGSGSGPRLAERKGRVGAARRQSPRAPGGGLPGPRSGPAAAFIRRRGRWPGPRCAPATPRPRSRLCAPTRLCLDEPSSVLCAGLEHQLTSDHCQPTPSHPRRSHLWASGIKQVLGCTMISPFLVLAIGTCLTNSLVPEKEKDPKYWRDQAQETLKYALELQKLNTNVAKNVIMFLGDGMGVSTVTATRILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLVHNIRDIDVIMGGGRKYMYPKNKTDVEYEIDEKARGTRLDGLDLVNIWKSFKPRHKHSHFIWNRTELLTLDPHNVDYLLGLFEPGDMEYELNRNNVTDPSLSEMVVVAIQILRKNPKGFFLLVEGGRIDHGHHEGKAKQALHEAVEMDRAIGQAGSMTSLEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYIPHVMAYAACIGANLDHCAPASSAGSLAAGPLLLPLALFPLSILF

SEQ ID NO: 8 - protein, 524 aa, Rattus norvegicus
MILPFLVLAIGPCLTNSFVPEKEKDPSYWRQQAQETLKNALKLQKLNTNVAKNIIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMRPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNPKGFFLLVEGGRIDHGHHEGKAKQALHEAVEMDEAIGKAGTMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWASSASSPSPGALLLPLALFPLRTLF

SEQ ID NO: 9 - protein, 502 aa, Canis lupus familiaris
EKDPKYWRDQAQQTLKYALRLQNLNTNVAKNVIMFLGDGMGVSTVTATRILKGQLHHNPGEETRLEMDKFPYVALSKTYNTNAQVPDSAGTATAYLCGVKANEGTVGVSAATQRTHCNTTQGNEVTSILRWAKDAGKSVGIVTTTRVNHATPSAAYAHSADRDWYSDNEMPPEALSQGCKDIAYQLMHNVKDIEVIMGGGRKYMFPKNRTDVEYEMDEKSTGARLDGLNLIDIWKNFKPRHKHSHYVWNRTELLALDPYTVDYLLGLFDPGDMQYELNRNNVTDPSLSEMVEIAIKILSKKPRGFFLLVEGGRIDHGHHEGKAKQALHEAVEMDRAIGKAGVMTSLEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANQDHCASASSAGGPSPGPLLLLLALLPVGILF

SEQ ID NO: 10 - protein, 252 aa, Sus scrofa
AELLALDPHTVDYLLGLFEPGDMQYELNRNNVTDPSLSEMVEMAIRILIKNPKGFFLLVEGGRIDHGHHEGKAKQALHEAVEMDRAIEQAGSMTSVEDTLTVVTADHSHVFTFGGYTPRGNSIFGLAPMVSDTDKKPFTAILYGNGPGYKVVGGERENVSMVDYAHDNYQAQSAVPLRHETHGGEDVAIFARGPMAHLLHGVHEQNYIPHVMAYAACVGANRDHCASASSSGSPSPGPLLLLLALLPLGILF

SEQ ID NO: 11 - protein, 524 aa, Mus musculus
MISPFLVLAIGTCLTNSFVPEKERDPSYWRQQAQETLKNALKLQKLNTNVAKNVIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMPPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNLKGFFLLVEGGRIDHGHHEGKAKQALHEAVEMDQAIGKAGAMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWAGSGSAPSPGALLLPLAVLSLRTLF

SEQ ID NO: 12 - protein, 524 aa, Bos taurus
MISPFLLLAIGTCFASSLVPEKEKDPKYWRDQAQQTLKNALRLQTLNTNVAKNVIMFLGDGMGVSTVTAARILKGQLHHSPGEETKLEMDKFPYVALSKTYNTNAQVPDSAGTATAYLCGVKANEGTVGVSAATQRSQCNTTQGNEVTSILRWAKDAGKSVGIVTTTRVNHATPSASYAHSADRDWYSDNEMPPEALSQGCKDIAYQLMHNIKDIEVIMGGGRKYMFPKNRTDVEYELDEKARGTRLDGLNLIDIWKSFKPKHKHSHYVWNRTDLLALDPHSVDYLLGLFEPGDMQYELNRNNATDPSLSEMVEMAIRILNKNPKGFFLLVEGGRIDHGHHEGKAKQALHEAVEMDQAIGQAGAMTSVEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANRDHCASASSSGSPSPGPLLLLLALLPLGSLF

SEQ ID NO: 13 - protein, 524 aa, Bos taurus; identical to SEQ ID NO: 12

SEQ ID NO: 14 - protein, 124 aa, Bos taurus
DPKYWRDQAQQTLKNALGLQKLNTKVAKNVILFLGDGMGVSTVTAARILKGQLHHNPGEETRLEMDKFPFVALSKTYNTNAQVPDSAGTAPHPVRVKAMRAPWGEPHQRQCNTRRATSTHLLAG

SEQ ID NO: 15 - protein, 524 aa, Felis catus
MISPFLVLAIGTCLTNSLVPEKEKDPKYWRDQAQQTLKNALRLQKLNTNVVKNVIMFLGDGMGVSTVTAARILKGQLHHNPGEETRLEMDKFPYVALSKTYNTNAQVPDSAGTATAYLCGVKANEGTVGVSAATQRTQCNTTQGNEVTSILRWAKDSGKSVGIVTTTRVNHATPSAAYAHSADRDWYSDNEMPPEALSQGCKDIAYQLMHNVRDIEVIMGGGRKYMFPKNRTDVEYEMDEKARGTRLDGLNLVDIWKSFKPRHKHSHYVWNRTELLTLDPYGVDYLLGLFEPGDMQYELNRNSTTDPSLSEMVEIAIKILSKNPKGFFLLVEGGRIDHGHHEGKAKQALHEAVEMDQAIGRAGAMTSVEDTLTIVTADHSHVFTFGGYTPRGNSIFGLAPMVSDTDKKPFTSILYGNGPGYKVVGGERENVSMVDYAHNNYQAQSAVPLRHETHGGEDVAVFAKGPMAHLLHGVHEQNYIPHVMAYAACIGANLDHCASASSAGGPSPGPLFLLLALPSLGILF

SEQ ID NO: 16 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 17 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 18 - protein, 532 aa, Homo sapiens; identical to SEQ ID NO: 16

SEQ ID NO: 19 - protein, 528 aa, Homo sapiens
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 20 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK